(19) United States
(12) Patent Application Publication
(10) Pub. No.: US 2010/0168044 A1
(43) Pub. Date: Jul. 1, 2010

(54) SUPERPARAMAGNETIC NANOPARTICLE ENCAPSULATED WITH STIMULI RESPONSIVE POLYMER FOR DRUG DELIVERY

(76) Inventor: Devesh Kumar Misra, Lafayette, LA (US)

Correspondence Address: KEAN, MILLER, HAWTHORNE, D'ARMOND,;MCCOWAN & JARMAN, L.L.P., ONE AMERICAN PLACE, 22ND FLOOR, P.O. BOX 3513, BATON ROUGE, LA, 70821, US

(21) Appl. No.: 12/344,404

(22) Filed: Dec. 26, 2008

(51) Int. Cl. A61K 47/48; A61K 31/704; A61K 31/519

(52) U.S. Cl. 514/34; 514/769; 514/249; 977/773; 977/906

(57) ABSTRACT

The current invention is a novel superparamagnetic site-targeting nanoparticle comprising superparamagnetic nanoparticles encapsulated with a smart polymer. The superparamagnetic site-targeting nanoparticle comprises a functionalized superparamagnetic core that is conjugated with a therapeutic agent and then encapsulated with a smart polymer. The smart polymer can be any polymer that exhibits a reversible conformational or physio-chemical change in response to an external stimulus or stimuli.

### CROSS REFERENCE TO RELATED APPLICATIONS

Not Applicable.

### STATEMENT REGARDING FEDERALLY SPONSORED RESEARCH OR DEVELOPMENT

Not Applicable.

### REFERENCE TO A “SEQUENCE LISTING,” A TABLE, OR A COMPUTER PROGRAM

Not Applicable.

### BACKGROUND OF THE INVENTION

1. Field of the Invention

The present invention relates to superparamagnetic nanoparticles encapsulated with a stimuli-responsive smart polymer, designed for the targeting and controlled release of a therapeutic agent.

2. Description of Related Art

There is currently significant interest in designing new drug delivery systems with the objective of achieving targeted drug delivery. Targeted drug delivery decreases the possible harmful side effects that many drugs exhibit because the targeted delivery of the drug decreases the interaction between the drug and non-targeted sites. Superparamagnetic nanoparticles have been researched for their use as drug-targeting carriers.

Surface modified superparamagnetic nanoparticles, which are characterized by the absence of magnetism on the removal of a magnetic field, have been intravenously delivered to the tumor site using an external magnetic field as described by A. Ito, M. Shinkai, H. Honda, and T. Kobayashi, ‘Medical application of functionalized magnetic nanoparticles,’ Journal of Bioscience and Bioengineering, vol. 100, 1-11 (2005), which in incorporated herein by reference. However, the practicality of using superparamagnetic nanoparticles for drug delivery applications has been reduced because generally, the superparamagnetic nanoparticles are rapidly cleared by macrophages or the reticuloendothelial system before they reach the desired therapeutic agent release site as disclosed by A. K. Gupta and M. Gupta, ‘Synthesis and surface engineering of iron oxide nanoparticles for biomedical applications, Biomaterials, vol. 26, 3995-4021 (2005), which is incorporated herein by reference. Additionally, non-surface modified superparamagnetic nanoparticles having large surface-area-to-volume ratios tend to agglomerate and form large clusters, resulting in the loss of their superparamagnetic characteristics.

Stimuli-responsive materials and molecules have numerous possible applications in the biomedical/pharmaceutical field, as well as in biotechnology and related industries. Smart conjugates, smart surfaces, smart polymeric micelles, and smart hydrogels have all been studied for a variety of diagnostics, separations, cell culture, drug delivery, and bioprocess applications.

Despite the development of superparamagnetic nanoparticle technologies for drug delivery applications, there exists a need for a biodegradable superparamagnetic nanoparticles that are not rapidly cleared by macrophages or the reticuloendothelial system before they reach the targeted site, can deliver an anti-tumor agent to the targeted site, retain their superparamagnetic characteristics, and exhibit a controlled release of the therapeutic agent once the superparamagnetic nanoparticles reach the targeted site.

It is an object of the present invention to provide a superparamagnetic nanoparticle therapeutic agent carrier which is biodegradable and exhibits a controlled release of a therapeutic agent.

It is an additional object of the present invention to provide a superparamagnetic nanoparticle therapeutic agent carrier which has the attributes described above as well as a stimuli-responsive therapeutic agent release.

It is a further object of the present invention to provide a superparamagnetic nanoparticle encapsulated with a biodegradable, stimuli-responsive polymer that has superparamagnetic and tumor targeting characteristics.

### SUMMARY OF THE INVENTION

One aspect of the present invention provides for a superparamagnetic nanoparticle comprising: (a) a core having responsivity to a magnetic field, wherein said core comprises a surface; (b) a therapeutic agent conjugated to said surface of said core; and (c) a stimuli-responsive polymer encapsulating said core and said therapeutic agent. In one embodiment of the present invention, said core comprises Fe₃O₄. In another embodiment of the present invention, said surface of said core comprising Fe₃O₄ is functionalized with —NHNH₂. In one embodiment of the present invention, said stimuli-responsive polymer comprises a biodegradable polymer. Another embodiment provides that said therapeutic agent comprises doxorubicin. Said stimuli-responsive polymer may comprise dextran-g-poly(NIPAAm-co-DMAAm) conjugated with folic acid. Additionally, said stimuli-responsive polymer may comprise chitosan-g-poly(NIPAAM-co-DMAAm). Said chitosan-g-poly(NIPAAM-co-DMAAm) stimuli-responsive polymer may also be conjugated with folic acid.

Another aspect of the present invention provides for a method for making superparamagnetic nanoparticles, comprising the steps: (a) making a nanoparticle core having responsivity to a magnetic field, wherein said nanoparticle core comprises a surface; (b) functionalizing said surface of said nanoparticle core; (c) conjugating a therapeutic agent to said surface of said functionalized nanoparticle core; and (d) encapsulating said conjugated therapeutic agent and said nanoparticle core with a stimuli-responsive polymer. In another embodiment of the present invention, said therapeutic agent comprises doxorubicin. Additionally, said stimuli-responsive polymer may comprise a biodegradable polymer. Said stimuli-responsive polymer may comprise dextran-g-poly(NIPAAm-co-DMAAm) conjugated with folic acid. In another embodiment of the present invention, said stimuli-responsive polymer comprises chitosan-g-poly(NIPAAM-co-DMAAm). Said chitosan-g-poly(NIPAAM-co-DMAAm) stimuli-responsive polymer may also be conjugated with folic acid.

A third aspect of the present invention provides a method for making a superparamagnetic nanoparticle encapsulated with a stimuli-responsive polymer, comprising the steps: (a) making a nanoparticle core having responsivity to a magnetic field, wherein said nanoparticle core comprises a surface; (b) functionalizing said surface of said nanoparticle core with a —NHNH₂ functional group; (c) conjugating a therapeutic agent to said surface of said functionalized nanoparticle core; and (d) encapsulating said conjugated therapeutic agent and said functionalized nanoparticle core with a stimuli-responsive polymer. In an another embodiment of the present invention, said therapeutic agent comprises doxorubicin. In one embodiment of the present invention, said stimuli-responsive polymer comprises a biodegradable polymer. Said stimuli-responsive polymer may comprise dextran-g-poly(NIPAAm-co-DMAAm) conjugated with folic acid. Additionally, said stimuli-responsive polymer may comprise chitosan-g-poly(NIPAAM-co-DMAAm).

The present invention has several advantages over the prior art systems. One advantage of the present invention is that the nanoparticle therapeutic agent carriers are encapsulated with a biodegradable stimuli-responsive smart polymer, designed for controlled release and both superparamagnetic and site-targeting characteristics.

Additionally, because the nanoparticles are superparamagnetic, the nanoparticles can be targeted to a specific site by targeting a magnetic field on the targeted site. In a preferred embodiment of the present invention, the nanoparticles are targeted to a tumor site. The external magnetic field heats the cells at the treatment site resulting in cell death, and thereby destroying the tumor cells.

Yet another advantage is that the nanoparticle anti-tumor agent carriers provide a non-invasive technique for treating cancer. Additionally, by using a targeted anti-tumor agent delivery system, the possibility of harmful side effects, which many anti-tumor drugs exhibit, is decreased because the targeted delivery of the anti-tumor agent decreases the interaction between the anti-tumor agent and non-tumor cells.

These and other objects, advantages, and features of this invention will be apparent from the following description.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a schematic illustration of the preferred mode for preparing the superparamagnetic nanoparticles of the present invention.

FIG. 2 is an illustration of the chemical conjugation of an anti-tumor agent, doxorubicin, to the surface of a nanoparticle.

FIG. 3a is a transmission electron micrograph (TEM) of the magnetite nanoparticles encapsulated with dextran-based smart polymer.

FIG. 3b is the selected area electron diffraction (SAED) pattern of the magnetite nanoparticles encapsulated with dextran-based smart polymer.

FIG. 4a is a TEM of the magnetite nanoparticles encapsulated with chitosan-based smart polymer.

FIG. 4b is the SAED pattern of the magnetite nanoparticles encapsulated with chitosan-based smart polymer.

FIG. 5 is a Fourier transform infrared spectroscopy (FTIR) spectra of the hydrophobic Fe₃O₄ nanoparticles, functionalized with a hydrazide end group, conjugated with doxorubicin, and encapsulated with dextran-based smart polymer.

FIG. 6 is a FTIR spectra of the hydrophobic Fe₃O₄ nanoparticles, functionalized with a hydrazide end group, conjugated with doxorubicin, and encapsulated with chitosan-based smart polymer.

FIG. 7 is a high-resolution transmission electron micrograph of functionalized Fe₃O₄ nanoparticles conjugated with doxorubicin and encapsulated with dextran-g-poly(NIPAAm-co-DMAAm) smart polymer.

FIG. 8 is a thermogravimetric analysis (TGA) curve of nanoparticles encapsulated with the dextran-based smart polymer.

FIG. 9 is a FTIR spectra of the chitosan-based smart polymer.

FIG. 10 is a high-resolution transmission electron micrograph of the functionalized Fe₃O₄ nanoparticles conjugated with doxorubicin and encapsulated with chitosan-based smart polymer.

FIG. 11 is a TGA curve of nanoparticles encapsulated with the chitosan-based smart polymer.

FIG. 12 is a picture of dextran-encapsulated nanoparticles of the present invention showing that the encapsulated nanoparticles are attracted by a magnet.

FIG. 13 is a ¹H NMR spectra of (a) poly(NIPAAm-co-DMAAm) and (b) dextran-g-poly(NIPAAm-co-DMAAm).

FIG. 14 is a graph depicting the lower critical solution temperature (LCST) of an aqueous solution of the dextran-based smart polymer containing 5 wt. % PBS solution (pH 7.4).

FIG. 15 is a graph depicting the LCST of an aqueous solution of the chitosan-based smart polymer containing 5 wt. % PBS solution.

FIG. 16 is a graph depicting the percentage cumulative doxorubicin release from the nanoparticle encapsulated with dextran-based smart polymer at temperatures of 20, 37, and 40° C. in PBS, at pH 7.4 and 5.3. The data points are the average of at least three experiments. The bars represent the range over which the values were observed.

FIG. 17 is a graph depicting the rate of doxorubicin release (mg h⁻¹) from the nanoparticle encapsulated with dextran-based smart polymer at temperatures of 20, 37, and 40° C. in PBS, at pH 7.4 and 5.3. The data points are the average of at least three experiments. The bars represent the range over which the values were observed.

FIG. 18 is a graph depicting the rate of doxorubicin release (mg h⁻¹) from bare nanoparticles and nanoparticles encapsulated with dextran-based smart polymer at temperatures of 20, 37, and 40° C. in PBS, at pH 5.3. The data points are the average of at least three experiments. The bars represent the range over which the values were observed.

FIG. 19 is a graph depicting the percentage cumulative doxorubicin release from bare nanoparticles and nanoparticles encapsulated with dextran-based smart polymer at 20 and 40° C. in PBS, at pH 5.3. The data points are the average of at least three experiments. The bars represent the range over which the values were observed.

FIG. 20 is a graph depicting the percentage cumulative doxorubicin release from nanoparticles encapsulated with chitosan-based smart polymer at temperatures of 20, 37, and 40° C. in PBS, at pH 7.4 and 5.3. The data points are averages of at least three experiments. The bars represent the range over which the values were observed.

FIG. 21 is a graph depicting the rate of doxorubicin release (mg h⁻¹) from nanoparticles encapsulated with chitosan-based smart polymer at temperatures of 20, 37, and 40° C. in PBS, at pH 7.4 and 5.3. The data points are averages of at least three experiments. The bars represent the range over which the values were observed

### DETAILED DESCRIPTION OF THE INVENTION

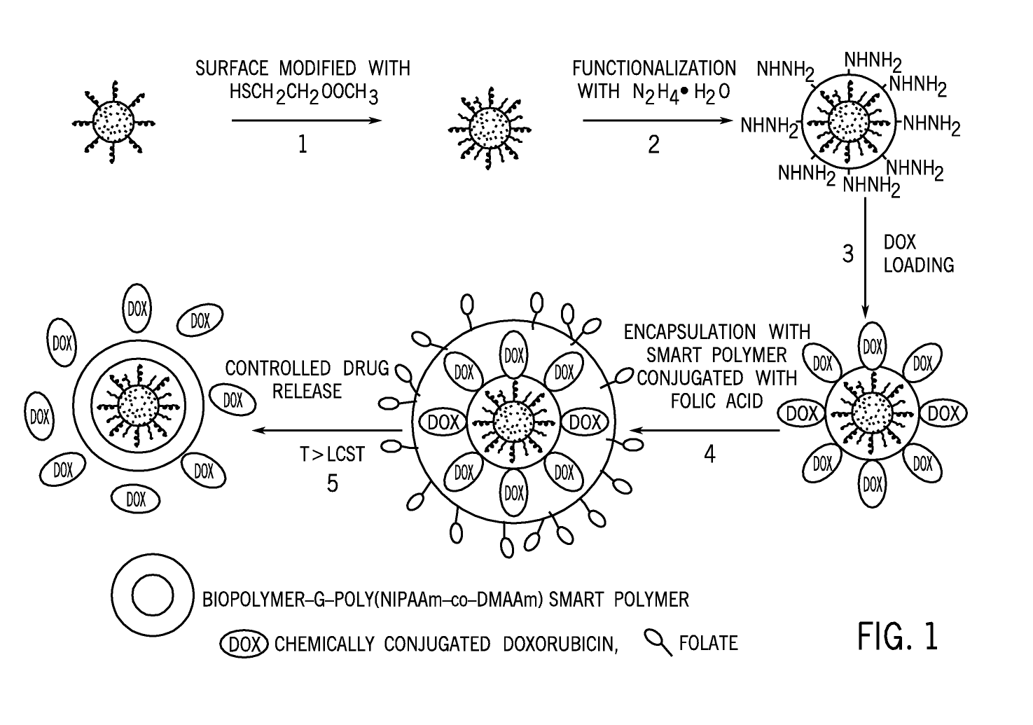

The present invention provides a superparamagnetic nanoparticle, having a therapeutic agent adsorbed onto the surface of the nanoparticle, encapsulated with a biodegradable stimuli-responsive smart polymer, as well as methods for making these nanoparticle therapeutic agent carriers. FIG. 1 depicts a preferred mode of preparing the superparamagnetic nanoparticles of the present invention. As shown in FIG. 1 step 1, first a superparamagnetic nanoparticle is surface modified with HSCH₂CH₂OOCH₃. Then the surface modified superparamagnetic nanoparticle is functionalized with N₂H₄.H₂O (FIG. 1, step 2). Next, the functionalized superparamagnetic nanoparticle is loaded with an anti-tumor agent (FIG. 1, step 3). The anti-tumor agent loaded superparamagnetic nanoparticle is then encapsulated with a smart polymer (FIG. 1, step 4). In a preferred embodiment of the present invention, the total size of the nanoparticle, after encapsulation with smart polymer, is approximately 8 nanometers (nm).

Nanoparticle Core. The nanoparticle of the present invention comprises a superparamagnetic core that has responsivity to a magnetic field. Suitable materials having responsivity to a magnetic field include, but are not limited to: nickel ferrite, NiFe₂O₄, cobalt ferrite, zinc ferrite, and any other magnetic particle. Nickel ferrite is not preferred as it is toxic. In a preferred embodiment of the present invention, the core is magnetite (Fe₃O₄). In a preferred embodiment of the present invention, the nanoparticle core is approximately 5 nm.

The core of the nanoparticles can be prepared several ways. In the preferred embodiment of the present invention, the cores of the nanoparticles are prepared using the reverse micelle technique as described in U.S. patent application Ser. No. 11/051,273, which is incorporated herein by reference. This approach offers substantial control over the size and size-distribution of the particles because the reaction occurs inside nanoreactors. Briefly, two microemulsion systems are prepared. The first system comprising an oil-phase microemulsion comprising isooctane and a suitable surfactant, such as diiso-octylsulphoccinate (AOT). The second system is an aqueous phase emulsion comprising isooctane and surfactant AOT with the reactant salts (hydrated iron sulfate). The first microemulsion system typically comprises 2 ml of 30% NH₄OH (precipitating agent)+2.4 ml of water+66 ml of 0.50 M AOT-isooctane. The second microemulsion system comprises 0.576 g of FeSO₄.7H₂O dissolved in 8 ml of water+66 ml of AOT-isooctane. Prior to use, both emulsion systems are separately sonicated for 10 minutes. The two microemulsions are subjected to rapid mechanical stirring for 75 minutes at a temperature of 50° C. Atomic force microscope (AFM) shows that at this temperature, particles with reduced roughness and high saturation magnetization are obtained. R. D. K. Misra, S. Gubbala, A. Kale and W. F. Egelhoff Jr., ‘A comparison of the magnetic characteristics of nanocrystalline nickel, zinc, and manganese ferrites synthesized by reverse micelle technique,’ Materials Science and Engineering B, vol. 111, 164-174 (2004), which is incorporated by reference. The iron hydroxide is precipitated within the water phase of the reverse micelles and oxidized to magnetite. The precipitation of Fe₃O₄ occurs according to the following reaction: 3FeSO₄.7H₂O+6NH₄OH+½O₂→Fe₃O₄↓3(NH₄)₂SO₄+24H₂O. After rapid mechanical stirring, methanol is added to the resulting mixture, to extract the surfactant and the organic solvent. The resulting liquid is separated and the magnetite product is centrifuged with more methanol. The resulting solid product is washed at least three times with 50% methanol and acetone mixture and distilled water, and dried in an oven at 90° C. for 30 minutes.

The core of the nanoparticles of the present invention has a diameter ranging from approximately 3 nanometers to approximately 8 nanometers, preferably between 5 nm and 8 nm. In this size range the cores of the nanoparticles are superparamagnetic, meaning that they do not exert an overall magnetic field until an external field is applied. If the particle size is too large, the nanoparticles will lose their superparamagnetic properties.

The nanoparticle core is then functionalized so that a therapeutic agent, preferably an anti-tumor agent, can be conjugated to the core. The nanoparticle is functionalized by forming a chemical bond between the nanoparticle core and the therapeutic agent. If the nanoparticle core is Fe₃O₄, the core is hydrophobic and therefore requires surface functionalization to enable its use for superparamagnetic drug targeting. The as-synthesized Fe₃O₄ nanoparticles are hydrophobic because of the attachment of oleylamine on the surface. Oleylamine was added during synthesis to ensure that the particles are individually dispersed. In a preferred embodiment of the present invention, the superparamagnetic core is surface functionalized to allow conjugation with an anti-tumor agent. The surface of the hydrophobic Fe₃O₄ nanoparticles was modified with bifunctional methyl-3-mercaptopropionate (HSCH₂CH₂COOCH₃), which was chemically bonded to the surface of the magnetite nanoparticles via Fe—S covalent bonds. To enable conjugation with an anti-tumor agent, such as doxorubicin, the —OCH₃ group is converted to the —NHNH₂ functional group by a hydrazinolysis reaction because the —NHNH₂ functional group facilitates the subsequent conjugation of a drug such as doxorubincin. Functionalization of the magnetite nanoparticle cores with the —NHNH₂ functional groups is achieved using a chemically bonded reaction via Fe—S covalent bonds, followed by hydrazinolysis reaction. The hydrazinolysis reaction provides hydrozide end groups (hydrazone linkage with anti-tumor agent) that are acid-labile hydrazone linkers with the ability to increase the rate of anti-tumor agent release in the acidic environment (e.g. pH approximately 5) present in the endosome or lysosome of cancerous cells The cleavage of the anti-tumor agent that is chemically bound to the functional groups depends on the type of linkage, notably peptide, hydrazone, and cisaconityl linkages

After the nanoparticle cores have been functionalized, a therapeutic agent is conjugated to the core. In a preferred embodiment, the therapeutic agent is an anti-tumor agent. In the most preferred embodiment of the present invention, the therapeutic agent is the anti-tumor drug, doxorubicin. Doxorubicin is an antineoplastic agent commonly used to treat tumors. Other drugs can be used as the therapeutic agent or anti-tumor agent. If other drugs are used, the functional groups on the superparamagnetic nanoparticles will need to be modified such that a chemical bond is formed between the drug and the superparamagnetic nanoparticle. Once a particular drug is chosen, then the functional group that is needed will be able to be determined by steps that are well known to one of ordinary skill in the art.

Stimuli-Responsive Polymers. Stimuli-responsive (also termed “intelligent” or “smart”) materials and molecules exhibit abrupt property changes in response to small changes in external stimuli such as pH; temperature; UV-visible light; ionic strength; the concentration of certain chemicals, such as polyvalent ions, polyions of either charge, or enzyme substrates, such as glucose; as well as upon photo-irradiation or exposure to an electric field. Usually these changes are fully reversible once the stimulus has been removed.

The stimuli-responsive polymers used in the present invention may be synthetic or natural polymers that exhibit reversible conformational or physico-chemical changes such as folding/unfolding transitions or other conformational changes, such as a change from hydrophilic to hydrophobic, in response to stimuli, such as to changes in temperature or pH. Stimuli-responsive polymers useful in making the nanoparticles of the present invention can be any stimuli-responsive polymer that is sensitive to a stimulus, meaning that the stimulus causes significant conformational changes in the polymer.

In a preferred embodiment of the present invention, the stimuli-responsive polymer can be any one of a variety of polymers that exhibit reversible conformational or physico-chemical changes to either of the external stimuli of pH or temperature. For example, a temperature-responsive polymer is responsive to changes in temperature by exhibiting a LCST in aqueous solution. The stimuli-responsive polymer can be a multi-responsive polymer, where the polymer exhibits property changes in response to combined simultaneous or sequential changes in two or more external stimuli. In a preferred embodiment of the present invention, the stimuli-responsive polymer is biodegradable.

The stimuli-responsive polymers useful in the nanoparticles of the present invention include copolymers having stimuli-responsive behavior. Other suitable stimuli-responsive polymers include graft copolymers having one or more stimuli-responsive polymer components. For example, a suitable stimuli-responsive graft copolymer may include a temperature-sensitive backbone and pH-sensitive polymer components.

The stimuli-responsive polymer can include a polymer having a balance of hydrophilic and hydrophobic groups, such as polymers and copolymers of N-isopropylacrylamide. In a preferred embodiment of the present invention, the stimuli-responsive polymer is a temperature responsive polymer, poly(N-isopropylacrylamide) (also referred to as “PNIPAAm” and “poly(NIPAAm)”).

PNIPAAm homopolymer and its copolymer are typical examples of thermosensitive polymers. Poly(N-isopropylacrylamide) and its copolymer are characterized by a lower critical solution temperature (LCST) in aqueous solution such that their volume and shape change in a reversible manner in response to small changes in temperature around the LCST. Accordingly, they experience a sharp coil-globule phase transition in water at the LCST, transforming from an expanded hydrophilic structure below the LCST to a compact hydrophobic structure above it. This property is due to the thermally-reversible interaction of water molecules with the hydrophobic groups, especially the isopropyl groups, leading to low entropy, hydrophobically-bound water molecules below the LCST and release of those water molecules at and above the LCST. Modification of superparamagnetic nanoparticles with poly(NIPAAm) yields superparamagnetic nanoparticles that are temperature-responsive.

The LCST of the PNIPAAm homopolymer can be tuned to be above normal body temperature (37° C.) by incorporating co-monomer units, such as N,N-dimethylacrylamide as described by C. D. L. H. Alarcón, S. Pennadam, and C. Alexander, ‘Stimuli responsive polymers for biomedical applications,’ Chemical Society Reviews, vol. 34, 276-285 (2005), and B. Twaites, C. D. L. H. Alarcón, and C. Alexander, ‘Synthetic polymers as drugs and therapeutics,’ Journal of Materials Chemistry, vol. 15, 441-455 (2005), both of which are incorporated herein by reference. If an aqueous solution of polymer exhibits a particular phase below a specific temperature and experiences phase separation above this temperature, the polymer is considered to have a lower critical solution temperature (LCST), which represents the phase transition temperature. It can also be said to undergo a discontinuous phase change in water in the vicinity of LCST. This “smart” behavior of polymer with an on-off trigger mechanism is attractive in controlled drug delivery and biomedical applications.

In a preferred embodiment, the smart polymer for therapeutic agent release is a branched polymer modified through the addition of hydrophobic branches rather than a single homopolymer (poly(N-isopropylacrylamide), PNIPAAm). Branched polymers of this type also exhibit temperature-responsive behavior at “cloud point” temperature. The “cloud point” temperature often has a wide temperature range resulting from the broadening of phase transition.

In one embodiment of the present invention, the stimuli-responsive polymer is a graft copolymer with dual sensitivities to pH and temperature. In a more preferred embodiment of the present invention, the stimuli-responsive polymer is dextran-grafted PNIPAAm. In the most preferred embodiment of the present invention, the stimuli-responsive polymer is chitosan-grafted PNIPAAm.

Dextran is a natural polysaccharide that is a biodegradable polymer. A biodegradable encapsulation polymer enables the encapsulation polymer to degrade under physiological conditions without harmful effects or significant changes in the hydration-dehydration of the thermoresponsive polymer. Dextran grafted poly(N-isopropylacrylamide-co-N,N-dimethylacrylamide) that is derived from N-isopropylacrylamide includes the advantage of bio- and cyto-compatibility and exhibits “enzymatic degradation” upon temperature increase, as described by K. M. Huh, Y. Kumashiro, T. Ooya, and N. Yui, ‘New synthetic route for dextran graft copolymers containing thermo-responsive polymers.’ Polymer Journal, 33, 108-111 (2001) and C. Lemarchand, R. Gref and P. Couvreur, ‘Polysaccharide-decorated nanoparticles.’ European Journal of Pharmaceutics and Biopharmaceutics, 58, 327-341 (2004), each of which is incorporated herein by reference. Furthermore, PNIPAAm having a molecular weight of less than 40,000 Da is easily excreted from the body.

Chitosan-grafted PNIPAAm can offer not only biodegradability but also the potential of a pH-responsive hydrogel. These stimuli-response properties are advantageous for tumor targeting systems, where an external thermal stimulus, such as the application of a magnetic field, is applied to control the anti-tumor agent release and the pH stimulus response occurs due to the change in the physiological pH of 7.4 to the acidic endosomal pH of 5.5, which is present in tumor cells. Chitosan is a copolymer of N-acetyl glucosamine and D-glucosamine and is manufactured mainly from renewable crustacean shell, such as crab, shrimp and squid pen. The functional properties of chitosan are mainly dependent on the acetyl content and molecular weight, along with other important physico-chemical parameters. It is preferred that the chitosan be 80%-95% deacetylated. Although low, medium, and high molecular weight chitosan may be used (50 kD-80 kD), in a preferred embodiment of the present invention, a low molecular weight chitosan is used so that the superparamagnetic nanoparticles do not become bulky. Additionally, chitosan is able to chelate metal ions. Chitosan is preferred over dextran for the following reasons: (a) Chitosan has a natural ability to bind metal ions. (b) The presence of reactive amine groups in chitosan provides easier ligand attachment for targeted delivery than the hydroxyl groups of dextran. (c) Dextran is a water-soluble polymer and thus the coating can cause untimely release of encapsulated drug before the target site is reached. In this regard, the solubility of chitosan in mild acid (endosomal pH) overcomes this limitation to some extent. (d) The presence of antidextran antibodies in humans can limit the cellular uptake of dextran-coated particles and induce antibody-mediated cytotoxicity. (e) Dextran is an uncharged polymer and cannot adhere to the negatively charged phospholipid bilayer of cellular membranes as effectively as cationic chitosan based vesicles. (f) After the nanoparticles are taken up by the cells, the polymer coating of the nanoparticles must degrade to release the encapsulated drug for higher efficiency. The presence of lysozyme in cellular endocytosis helps to degrade chitosan, but does so to a lesser degree in the case of dextran, thus enhancing its biodegradability. The above numerated characteristics of chitosan provide for increased efficacy and lower toxicity for treating primary and advanced metastatic tumors.

Encapsulating the functionalized nanoparticle core conjugated with therapeutic agent with stimuli-responsive polymer. The functionalized nanoparticle core conjugated with the therapeutic agent is then encapsulated with the dextran-based stimuli-responsive polymer, or most preferred, the chitosan-based stimuli-responsive polymer. In a preferred embodiment of the present invention, the encapsulation layer is approximately 2-3 nm thick. In a preferred embodiment of the present invention, the cross-linked NIPAAm copolymer (hydrogel) is used as the encapsulated layer on the nanoparticle therapeutic agent carrier for controlling therapeutic agent release. However, the disadvantage of PNIPAAm homopolymer and its copolymer with N,N-dimethylacrylamide is that it is not biodegradable. Therefore in a most preferred embodiment of the present invention, the PNIPAAm is modified so that it is rendered biodegradable. The enzymatic degradation of PNIPAAm is promoted by grafting it with water-soluble and biodegradable dextran or chitosan, thus rendering PNIPAAm more biodegradable. Furthermore, as disclosed above, PNIPAAm with a molecular weight of less than 40,000 Da can be cleared or excreted by the physiological system. The grafted copolymer and its hydrogel that are formed by chemical cross-linking of the grafted dextran with 1,6-hexamethylenediamine cross-linker were observed to biodegrade via enzymatic reaction. The grafted dextran copolymers in phosphate-buffered solution (PBS) exhibited an LCST of approximately 40° C. because of the thermosensitive nature of the grafted polymer. The temperature of 40° C. is relevant because the application of a magnetic field will heat the nanoparticle, increasing the temperature from the physiological temperature of 37° C. to approximately 40-42° C. The increased temperature will likely kill cancerous cells. Additionally, in a manner similar to poly(ethylene glycol), encapsulating the nanoparticle with dextran may increase circulation time because dextran minimizes the interactions between the iron in magnetite and plasma proteins, leading to slower clearance from the intravascular system.

Conjugation with folic acid for tumor targeting. Folic acid conjugated polymers are used for tumor targeting. Folate is involved in the biosynthesis of amino acids and nucleic acids. Additionally, folate is a high-affinity ligand. Folate receptor is a tumor associated protein. Folic acid binds the folate receptors present on the tumor cells. Once the folic acid binds to the folate receptor, the nanoparticle is internalized through a process termed receptor-mediated endocytosis. Folate receptors appear to occur in low levels in most normal tissue. However, folate receptors are present at moderate to high levels in certain types of cancer. To increase the tumor targeting characteristics of the present invention, folic acid is conjugated to dextran-g-poly(NIPAAm-co-DMAAm) or chitosan-g-poly(NIPAAM-co-DMAAm) encapsulated drug-loaded superparamagnetic nanoparticles using N-hydroxysuccinimide (NHS) chemistry.

### EXAMPLES

Making Magnetite Core. Superparamagnetic Fe₃O₄ nanoparticles were synthesized using the high-temperature decomposition method. In a 50 milliliters (ml) three-neck flask, 20 ml of biphenyl ether, 0.71 grams (2 mmol) of iron(III) acetylacetonate, 2.25 grams (10 mmol) of 1,2-dodecanediol, 2.12 ml (6 mmol) of oleic acid and 2.19 ml (6 mmol) of oleylamine were intimately mixed by magnetic stirring. Oleylamine is added so that the nanoparticles are monodispersed. The synthetic reaction was carried out at 200° C. under nitrogen atmosphere for 2 hours and subsequently refluxed at approximately 260° C. for 1 hour in the absence of nitrogen. 40 ml of ethanol was added to the refluxed product, which yielded a black Fe₃O₄ precipitate. The black Fe₃O₄ precipitate was separated from the solution by centrifuging at 15,000 rpm for 30 minutes and washed at least 3 times with ethanol or until the rinse is clear.

Functionalizing Magnetite Core. To surface functionalize the magnetite nanoparticles, 138 milligrams (mg) of hydrophobic Fe₃O₄ nanoparticles were dispersed in 20 ml of diphenyl ether to form a colloid solution by sonication. Next, 33 microliters (μl) of methyl-3-mercaptopropionate was added to the colloid solution and then refluxed at approximately 259° C. for 1 hour. Subsequently, the solution was cooled to 100° C. Then 145 μl of hydrazine monohydrate (N₂H₄.H₂O) was added dropwise to the solution and then the solution was continuously stirred for 2 hours. The resulting nanoparticles were separated by centrifuging at 15,000 rpm for 10 minutes, washed three times with methanol and dried at approximately 50° C. for 24 hours. As a consequence of the above procedure, the hydrophilic Fe₃O₄ nanoparticles were functionalized by adding a —NHNH₂ group on the surface of the nanoparticles.

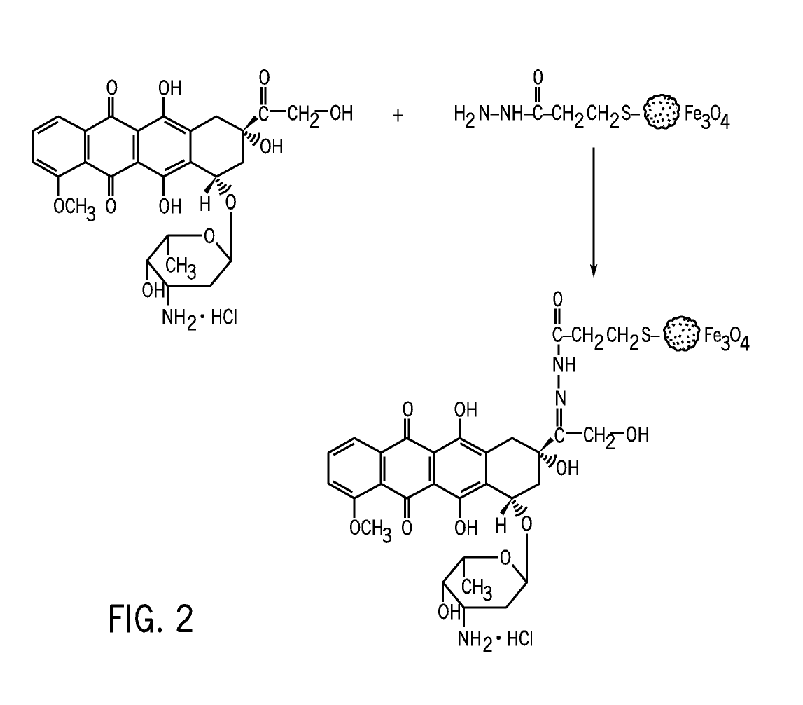

Fixing Doxorubicin on Nanoparticle. The anti-tumor drug doxorubicin was chemically adsorbed on the surface of the functionalized magnetite nanoparticles through an acid-labile hydrazone-bond, which is formed by the reaction of the hydrazide group of HSCH₂CH₂CONHNH₂ with the carbonyl group of doxorubicin. The desired amount (determined by the weight of the doxorubicin as a percentage of the weight of the entire nanoparticle) (e.g. 90-120 mg, W_{feed DOX}) of the hydrophilic Fe₃O₄ nanoparticles, which are now surface functionalized with a —NHNH₂ group, were dispersed by sonication in 20 ml of anhydrous methanol containing three drops of acetic acid, a catalyzer, to form a colloid solution. The amount of doxorubicin added to the colloid solution with continuous stirring was one-tenth the weight of the functionalized Fe₃O₄ nanoparticles. The reaction was carried out at room temperature for 48 hours. This procedure resulted in the chemical conjugation of doxorubicin (DOX) according to the reaction shown in FIG. 2.

The resulting colloidal solution was centrifuged at 15,000 rpm for 10 minutes. The precipitate obtained was redispersed in methanol by sonication, and centrifuged again. This process was repeated until the solution became colorless and particles settled at the bottom of the test tube. The DOX-loaded magnetite nanoparticles were then dried at approximately 50° C. for 24 hours. The centrifuged solution was collected and diluted to 100 ml with methanol in a capacitance flask. The free doxorubicin weight (W_{free DOX}) in the solution was determined by ultraviolet-visible (UV-Vis) spectrophotometry (Lauda Brinkmann, Germany) at wavelength 264 nm using the Lambert-Beer law, A=εcl, where A is the absorptance, ε is the molar absorptivity, c is the doxorubicin concentration and l is the path length of the quartz cell (1 centimeter). The DOX-loading efficiency was calculated as follows:

DOX-loading efficiency (%)=100(W_{feed DOX}−W_{free DOX})/W_{feed DOX} (Eq. 2)

The DOX-loading efficiency estimated using this above calculation was 89%.

Making Smart Polymer. A biodegradable, stimuli-responsive polymer was synthesized and its LCST was tuned to be slightly above normal body temperature (37° C.) by the following procedure. As described above, the biodegradable, stimuli-responsive polymers can be dextran based, or more preferred, chitosan based. The dextran-g-poly-(NIPAAm-co-DMAAm) smart polymer combines the stimuli-responsive behavior of poly(NIPAAm-co-DMAAm) polymer with the properties of enzymatic degradation of dextran using a grafted reaction method. Since the physiological pH in the blood stream is approximately 7.4 and the pH in the endosomes of some cancer cells is in the range of 5-5.5, Na₂HPO₄—KH₂PO₄ was used to buffer solutions with a pH of 7.4 and 5.3 as the drug release medium.

Dextran-Based Smart Polymer. The synthesis of the dextran-based smart polymer, involves the four stages:

(a) synthesis of the poly(NIPAAm-co-DMAAm) with a hydroxyl end-group [poly(NIPAAm-co-DMAAm)-COOCH₃];

(b) transformation of poly(NIPAAm-co-DMAAm)-COOCH₃ into poly(NIPAAm-co-DMAAm)-NHNH₂;

(c) activation of dextran with 4-nitrophenyl chloroformate, and

(d) synthesis of the dextran-g-poly(NIPAAm-co-DMAAm).

The synthesis reaction steps (a) and (b) are the free radical copolymerization and hydrazinolysis reactions, respectively. The resulting poly(NIPAAm-co-DMAAm)-NHNH₂ polymer was dialyzed against water using a dialysis membrane (MW 6 1,200) for 3 days and freeze-dried.

For dextran-based smart polymers, the activation reaction of dextran with 4-nitrophenyl chloroformate (step (c)) was carried out using 4-dimethylaminopyridine (DMAP) as a catalyzer. In a 250 ml solution of DMSO/pyridine (volume ratio 1/1) were dissolved 4.0 g of dextran, 4.35 g of 4-nitrophenyl chloroformate and 0.20 g of DMAP, and the resulting solution kept at 0° C. for 8 hours. The product was then precipitated in ethyl alcohol and filtered. The product was washed twice with ethyl alcohol, and then the product was dried at 50° C.

The synthesis of dextran-g-poly(NIPAAm-co-DMAAm) graft copolymer was performed by the coupling reaction involving 4-nitrophenyl chloroformate-activated dextran and poly(NIPAAm-co-DMAAm)-NHNH₂. First, 1.0 g of activated dextran and 0.76 g of poly(NIPAAm-co-DMAAm)-NHNH₂ were dissolved in 60 ml of DMSO and reaction carried out at room temperature for 48 hours. After precipitation in diethyl ether and drying at 50° C., the dried product was dialyzed (MW=12,400) against deionized water for 3 days to remove unreacted poly(NIPAAm-co-DMAAm)-NHNH₂ and then freeze-dried.

Chitosan-Based Smart Polymer. The synthesis of the chitosan-based biodegradable, stimuli-responsive polymer consisted of six stages: (a) synthesis of the poly(NIPAAm-co-DMAAm) with a hydroxyl end group [poly(NIPAAm-co-DMAAm)-COOCH₃]; (b) transformation of poly(NIPAAm-co-DMAAm)-COOCH₃ into poly(NIPAAm-co-DMAAm)-NHNH₂; (c) synthesis of organosoluble chitosan (N-phthaloylchitosan); (d) activation of N-phthaloyl-chitosan with 4-nitrophenyl chloroformate; (e) synthesis of the [N-phthaloyl-chitosan-g-poly(NIPAAm-co-DMAAm)]; and (f) removal of N-phthaloyl-chitosan. Steps (a) and (b) concern free radical copolymerization and hydrazinolysis reactions, respectively. The resulting poly(NIPAAm-co-DMAAm)-NHNH₂ polymer was dialyzed against water using a dialysis membrane (MW 6 1200) for 3 days and freeze-dried.

The organosoluble chitosan (N-phthaloyl-chitosan) (step (c)) was prepared by a phthaloylation reaction because N-phthaloyl-chitosan is a convenient precursor for chemical modification and has good solubility in solvents such as DMSO, which was used in the synthesis steps (d) and (e). Also, after the modification reaction, the N-phthaloyl group can be removed with hydrazine monohydrate (step (f)) to regenerate the free amino group. Organosoluble chitosan (N-phthaloyl-chitosan) was prepared using the process described below. 5.00 g of chitosan and 13.8 g of phthalic anhydride in 100 ml of DMF were heated with continuous stirring at 130° C. under an argon atmosphere. The solution was clear and viscous after about 6 hours. The precipitate was obtained by transferring the solution into 300 ml of ice-water and filtering. Subsequently, the precipitate was washed with ethanol and dried at 50° C. The activation of N-phthaloyl-chitosan with 4-nitrophenyl chloroformate (step (d)) was carried out using DMAP as a catalyzer. For this, 1.0 g of N-phthaloyl-chitosan, 4.35 g of 4-nitrophenyl chloroformate and 0.20 g of DMAP were dissolved in 250 ml of a DMSO/pyridine (1:1 v/v) solution and the resulting solution was kept at 0° C. for 8 hours. The product was precipitated in ethyl alcohol and filtered. After washing twice with ethyl alcohol, the product was dried at 50° C.

The synthesis of N-phthaloyl-chitosan-g-poly(NIPAAmco-DMAAm) (step (e)) graft copolymer was performed by the coupling reaction involving 4-nitrophenyl chloroformateactivated N-phthaloyl-chitosan and poly(NIPAAm-co-DMAAm)-NHNH₂. 0.84 g of activated N-phthaloyl-chitosan and 0.64 g of poly(NIPAAm-co-DMAAm)-NHNH₂ were dissolved in 60 ml of DMSO and the reaction was allowed to progress at room temperature for 48 hours. After precipitation in diethyl ether and drying at 50° C., the dried product was dialyzed (MW=12,400) against deionized water for 3 days to remove unreacted poly(NIPAAmco-DMAAm)-NHNH₂ and then freeze-dried.

Removal of N-phthaloyl (step (f)) was performed using the following procedure. A 200 mg suspension of N-phthaloyl-chitosan-g-poly(NIPAAM-co-DMAAm) in 20 ml of hydrazine monohydrate was stirred at 90° C. for 18 hours in a nitrogen atmosphere to remove the N-phthaloyl group. The reaction mixture was precipitated in diethyl ether. Subsequently, the precipitate was diluted with water and dialyzed (MW=12,400) against deionized water for 72 hours to collect chitosan-g-poly(NIPAAM-co-DMAAm).

Encapsulating Nanoparticle with Smart Polymer. The encapsulation of the superparamagnetic nanoparticles with the dextran-based biodegradable, stimuli-responsive polymer was accomplished using the following procedure: drug-loaded superparamagnetic nanoparticles, dextran-g-poly(NIPAAm-co-DMAAm) smart polymer and 1,6-diaminohexane (cross-linker), having a predetermined weight ratio of 2:3:1, were dispersed in 25 ml of dimethyl sulfoxide (DMSO). The encapsulation procedure involved cross-linking reaction with 1,6-diaminohexane, while sonicating for 2 hours at 40-50° C. Since the dextran-g-poly(NIPAAm-co-DMAAm) smart polymer contains the active 4-nitrophenyl chloroformate groups, it can be cross-linked with the superparamagnetic nanoparticles by 1,6-diaminohexane. After sonication, the Fe₃O₄ nanoparticles encapsulated with smart polymer were separated from the colloidal solution by centrifuging at 15,000 rpm for 20 minutes. They were then washed at least three times with methanol to remove any unreacted Fe₃O₄ nanoparticles and free dextran-g-poly(NIPAAm-co-DMAAm), and dried at approximately 50° C. for 24 hours. The weight percentage of drug present in the carrier was approximately 9%.

The encapsulation of the superparamagnetic nanoparticles with the chitosan-based biodegradable, stimuli-responsive polymer was achieved as follows: doxorubicin-loaded magnetite nanoparticles, chitosan-g-poly(NIPAAM-co-DMAAm) smart polymer and 1,6-diaminohexane (cross-linker) with a predetermined weight ratio of 2:3:1 were dispersed in 25 ml of DMSO and sonicated for 2 hours at 40-50° C. The encapsulated procedure involved a cross-linking reaction with 1,6-diaminohexane. Given that chitosan-g-poly(NIPAAM-co-DMAAm) biodegradable, stimuli-responsive polymer contains active 4-nitrophenyl chloroformate groups, they are cross-linked with the nanoparticles by 1,6-diaminohexane. After sonication, the magnetite nanoparticles encapsulated by smart polymer were separated from the colloidal solution by centrifuging at 26,893 g for 20 minutes. They were then washed at least three times with methanol to remove any unreacted molecules and any free chitosan-g-poly(NIPAAMco-DMAAm), and then dried at approximately 50° C. for 24 hours. The weight percentage of doxorubicin present in the carrier was estimated to be approximately 10%. This estimate was made based on the doxorubicin-loading efficiency calculated using Eq. (2) (above) and the pre-determined ratio of the doxorubicin-loaded nanoparticles and biodegradable, stimuli-responsive polymer.

The following examples indicate that the therapeutic agent release is dependent on a number of variables, such as particle size, surface properties, degradation rate, the interaction force of the drug binding to the surface, and the rate of hydration and dehydration of the thermosensitive polymers. The magnetite nanoparticle drug carrier encapsulated by a chitosan-grafted biodegradable, stimuli-responsive polymer appears to have two prime factors that determine the drug release response. The two prime factors are the LCST of the chitosan-based polymer and the binding affinity of the drug to the functionalized magnetite nanocarrier. In the experimental conditions below, the therapeutic agent release response for the magnetite nanoparticle drug carrier encapsulated by a chitosan-based polymer is influenced only by the triggered therapeutic agent release mechanism and pH. The therapeutic agent release rate, however, can be increased using a magnetic field, which can further reduce the duration of the controlled release.

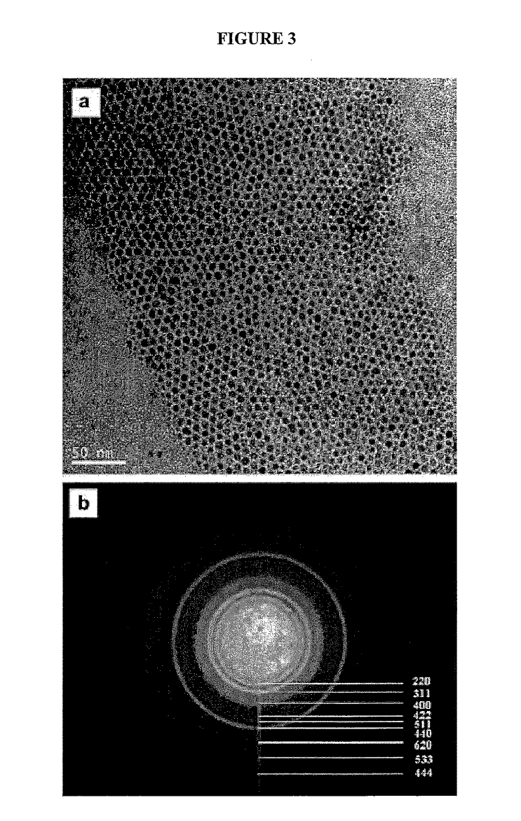

Morphology and structure of the magnetite nanoparticles. The sample preparation for examining the morphology, size range and structural characterization of the as-synthesized Fe₃O₄ nanoparticles and Fe₃O₄ nanoparticles conjugated with doxorubicin and encapsulated with the dextran-based smart-polymer was accomplished by dispersing them in hexane and deionized water, respectively. A drop of the liquid containing the dispersed nanoparticles was placed on a copper grid for study using a HITACHI H-7600 transmission electron microscope (TEM) at an accelerating voltage of 100 kV in conjunction with selected area electron diffraction (SAED). A transmission electron micrograph of the magnetite nanoparticles and the corresponding SAED pattern are presented in FIGS. 3a and 3b respectively. The monodisperse Fe₃O₄ nanoparticles have an average diameter of approximately 5 nm and are nearly spherical in shape. The results of indexing the SAED pattern are summarized in Table 1, together with the standard d-values listed in the X-ray powder JCPDS diffraction data file. A comparison of experimental and standard data suggests that the nanoparticles have a cubic crystal structure, with the d-values of magnetite being consistent with the standard values.

TABLE 1
A comparison of experimental and standard interplanar spacing (d) values
with their respective plane index (hkl) in Fe₃O₄ nanoparticles

| | d (nm), | D (nm), | hkl diffraction |
|---|---|---|---|
| Phase | experimental | standard [38] | plane |
| Fe₃O₄ | — | 4.86 | 111 |
| (cubic) | 2.96 | 2.97 | 220 |
| | 2.53 | 2.53 | 311 |
| | 2.09 | 2.10 | 400 |
| | 1.70 | 1.71 | 422 |
| | 1.61 | 1.62 | 511 |
| | 1.48 | 1.48 | 440 |
| | 1.32 | 1.33 | 620 |
| | 1.28 | 1.29 | 533 |
| | 1.21 | 1.21 | 444 |

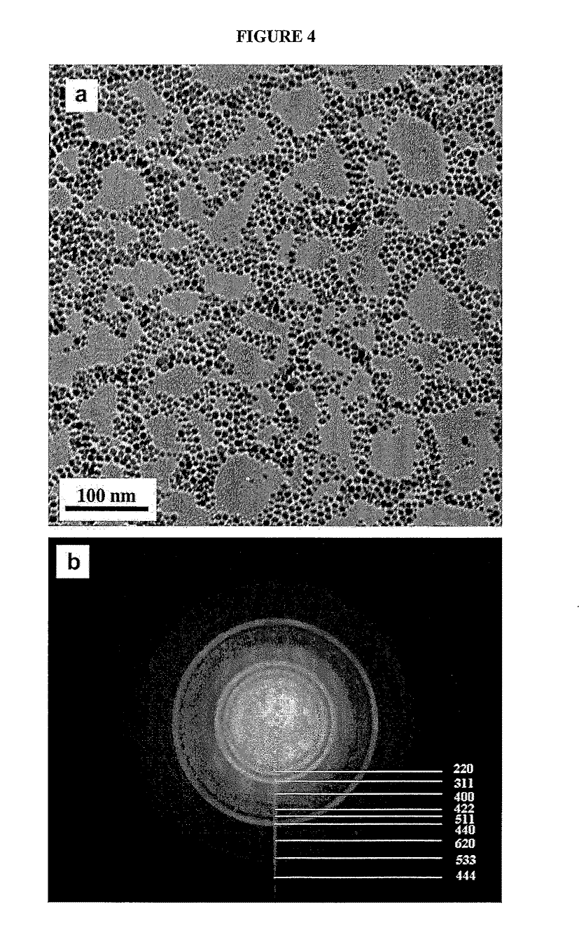

A transmission electron micrograph of the magnetite nanoparticles encapsulated with chitosan-based smart-polymer and the corresponding SAED pattern are presented in FIGS. 4a and 4b respectively. As shown in FIGS. 4a and 4b, the monodisperse Fe₃O₄ nanoparticles have an average diameter of approximately 5 nm and are nearly spherical in shape. The results of indexing the SAED pattern are summarized in Table 1 (above), together with the standard d-values listed in the X-ray powder JCPDS diffraction data file. A comparison of experimental and standard data suggests that the nanoparticles have a cubic crystal structure, with the d-values of magnetite being consistent with the standard values.

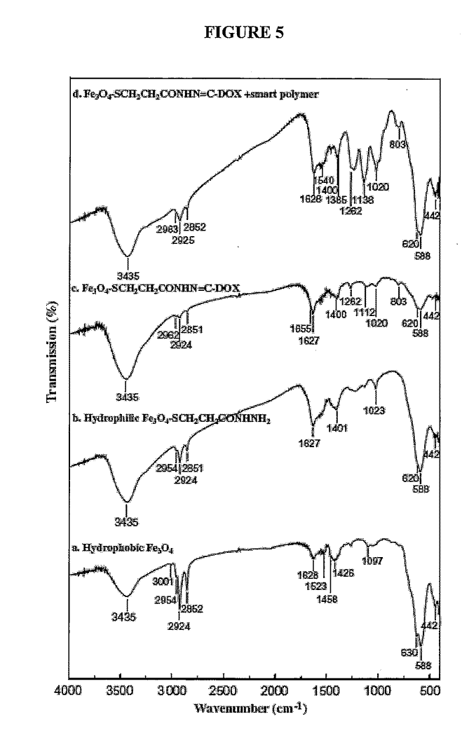

Surface functionalization and conjugation. The Fourier transform infrared spectroscopy (FTIR) technique provides information on chemical adsorption or chemical interaction. Thus, FTIR (FT/IR-480) spectra were obtained for different samples using a KBr compressed pellet method in the transmission mode at 4 cm⁻¹ resolution. After modifying the surface of the hydrophobic Fe₃O₄ nanoparticles by chemical bonding with bifunctional methyl-3-mercaptopropionate (HSCH₂CH₂COOCH₃) via Fe—S covalent bonds, the —OCH₃ functional group was subsequently converted to —NHNH₂ functional group by hydrazinolysis reaction. In this manner, the hydrophobic Fe₃O₄ nanoparticles were transformed into the hydrophilic Fe₃O₄ nanoparticles. The FTIR spectra of the hydrophobic Fe₃O₄ nanoparticles, functionalized with a hydrazide end-group, conjugated with doxorubicin and encapsulated with dextran-based smart polymer, are presented in FIG. 5. The assignments of the characteristic FTIR absorption bands presented in FIG. 5 are summarized in the Table 2 below.

TABLE 2

| Samples | IR absorption bands (cm⁻¹) | Description* |
|---|---|---|
| (a) Hydrophobic Fe₃O₄ nanoparticles | 3435 | ν(NH ··· H) |
| | 3001 | ν(═C—H) |
| | 2954, 2924, 2852 | νₐₛ(C—H) and νₛ(C—H) of —CH₂ |
| | 1628 | ν(C═O) |
| | 1523 | Absorption band of Fe—N bond |
| | 1458, 1426 | δₐₛ(CH₃) and δₛ(CH₃) |
| | 1097 | ν(C—N) |
| | 630, 588 | Absorption bands of the Fe—O bond |
| | 442 | Absorption bands of the Fe—O bond |
| (b) Hydrophilic Fe₃O₄—SCH₂CH₂CONHNH₂ | 3435 | ν(N—H ··· H) |
| | 2954, 2924, 2851 | νₐₛ(C—H) and νₛ(C—H) of —CH₂ |
| | 1627 | ν(C═O) |
| | 1401 | ν(—NH₃⁺) |
| | 1023 | ν(C—N) |
| | 620, 588 | Absorption bands of the Fe—O and Fe—S bonds |
| | 442 | Absorption bands of the Fe—O bond |
| (c) Fe₃O₄—SCH₂CH₂ CONHN═C-DOX | 3435 | ν(N—H ··· H) and ν(O—H ··· H) |
| | 2962, 2924, 2851 | νₐₛ(C—H) and νₛ(C—H) of —CH₃ and —CH₂ |
| | 1655 | ν(C═N—) |
| | 1627 | ν(C═O) |
| | 1400 | ν(—NH₃⁺) |
| | 1262 | ν(═C—O—CH₃) of doxorubicin |
| | 1112, 1020 | δ(O—H) |
| | 803 | Absorption band from doxorubicin |
| | 620, 588 | Absorption bands of the Fe—O and Fe—S bonds |
| | 442 | Absorption band of the Fe—O bond |
| (d) Fe₃O₄—SCH₂CH₂CONHN═C-DOX + | 3435 | ν(N—H ··· H) and ν(O—H ··· H) |
| smart polymer | 2963, 2925, 2852 | νₐₛ(C—H) and νₛ(C—H) |
| | 1628 | ν(C═O) |
| | 1540, 1385 | ν(—CH(CH₃)₂) of the smart polymer |
| | 1400 | ν(—NH₃⁺) |
| | 1262 | ν(═C—O—CH₃) of doxorubicin |
| | 1138, 1020 | δ(O—H) |
| | 803 | Absorption band from doxorubicin |
| | 620, 588 | Absorption bands of the Fe—O and Fe—S bonds |
| | 442 | Absorption band of the Fe—O bond |

*νₛ = symmetric stretching vibration; νₐₛ = asymmetric stretching vibration; δ = bending vibration.

FIG. 5a is the FTIR spectra of the hydrophobic bare Fe₃O₄ nanoparticle cores. The characteristic absorption bands at 3435 cm⁻¹ (ν(N—H)), 3001 cm⁻¹ (ν(=C—H)), 2954, 2924 and 2852 cm⁻¹ (νₐₛ(C—H), νₛ(C—H)), 1628 cm⁻¹ (ν(C═O)), 1458 and 1426 cm⁻¹ (δₐₛ(CH₃), δₛ(CH₃)), and 1097 cm⁻¹ (ν(C—N)) originate from oleylamine. The characteristic absorption bands of the hydrophobic Fe₃O₄ nanoparticles at 630, 588 and 442 cm⁻¹ are attributed to Fe—O bonds, while the absorption band at 1523 cm⁻¹ is assigned to absorption of Fe—N bonds, suggesting that —NH₂ coordinates with Fe(III) on the surface of the nanoparticles.

The FTIR spectra in FIG. 5b and assignment of their characteristic absorption bands listed in Table 2 imply successful functionalization of hydrophilic magnetite nanoparticles with HSCH2CH2CONHNH2 using a chemically bonded reaction via Fe—S covalent bonds and the hydrazinolysis reaction method. The characteristic absorption bands of FTIR spectra of Fe₃O₄—SCH₂CH₂CONHNH₂ (FIG. 5b) include ν(N—H) (3435 cm⁻¹), ν(C—H) (2954, 2924, 2851 cm⁻¹), ν(C═O) (1627 cm⁻¹), νas(-NH3⁺) (1401 cm⁻¹), ν(C—N) (1023 cm⁻¹) and ν(Fe—S, Fe—O) (620, 588 and 442 cm⁻¹). It may be noted from the FTIR spectra (FIG. 5b and Table 2) that most of oleylamine surfactant on the surface of the magnetite nanoparticles has been replaced by HSCH₂CH₂CONHNH₂.

In a manner similar to the confirmation of functionalization, the Fe₃O₄—SCH₂CH₂CONHN═C-DOX (doxorubicin) conjugate obtained via reaction of hydrazide groups of the Fe₃O₄—SCH₂CH₂CONHNH₂ with carbonyl groups of the doxorubicin was confirmed by FTIR (FIG. 5c). The 1655 cm⁻¹ characteristic absorption band is attributed to characteristic absorption of C═N bonds resulting from the reaction of hydrazide group of the Fe₃O₄—SCH₂CH₂CONHNH₂ with the carbonyl group of doxorubicin. The characteristic absorption bands of 803 and 1262 cm⁻¹ (ν(=C—O—CH₃ bonds)) correspond to doxorubincin, while the other characteristic absorption bands outlined above continue to be present (FIG. 5c). The above observations imply that doxorubicin is chemically conjugated on the surface of the functionalized magnetite nanoparticles.

The main characteristic absorption bands corresponding to dextran at 3700-3200 cm⁻¹ (ν(H—O . . . H)), 1138 and 1020 cm⁻¹ (δ(O—H)) and from the poly(NIPAAm-co-DMAAm) at 2963, 2925 and 2852 cm⁻¹ (νₐₛ(C—H) and νₛ(C—H) of —CH₃ and —CH₂) and 1540 and 1385 cm⁻¹ (ν(-CH(CH₃)₂)) were observed in FIG. 5d. This leads us to suggest that the functionalized magnetite nanoparticles conjugated with doxorubicin were successfully encapsulated with dextran-g-poly(NIPAAm-co-DMAAm) smart polymer to form drug-loading superparamagnetic nanoparticles encapsulated with dextran-based smart polymer. The nanoparticles are characterized by functionalized Fe₃O₄, which have the doxorubicin attached to the surface and are encapsulated with dextran-g-poly(NIPAAm-co-DMAAm) smart polymer.

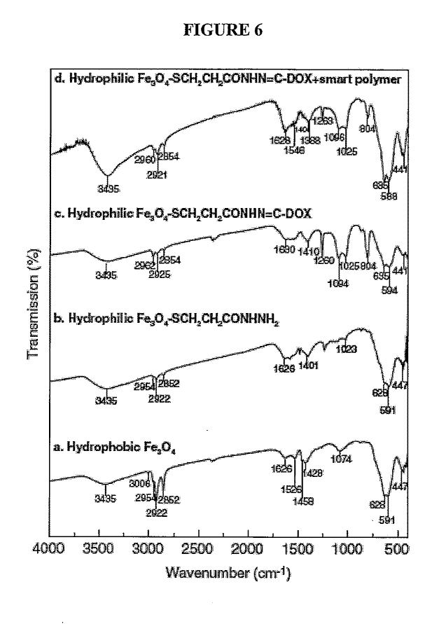

The results were similar for chitosan-based smart polymer encapsulation of the nanoparticles. FIG. 6a is the FTIR spectra of the bare hydrophobic Fe₃O₄ nanoparticles. The characteristic absorption bands at 3435 cm⁻¹ (ν(N—H)), 3006 cm⁻¹ (ν(=C—H)), 2954, 2922 and 2852 cm⁻¹ (νₐₛ(C—H), νₛ(C—H)), 1458 and 1428 cm⁻¹ (δₐₛ(CH₃), δₛ(CH₃)), and 1074 cm⁻¹ (ν(C—N)) are associated with the surfactant (oleylamine and oleic acid), while the 1628 cm⁻¹ (ν(C═O)) band originates form the oleic acid added during the synthesis of magnetite. The characteristic absorption bands of the hydrophobic Fe₃O₄ nanoparticles at 628, 591 and 447 cm⁻¹ are attributed to Fe—O bonds, while the absorption band at 1526 cm⁻¹ is assigned to the absorption of Fe—N bonds, suggesting that —NH₂ coordinates with Fe(III) on the surface of the nanoparticles.

The FTIR spectra in FIG. 6b and the assignment of the characteristic absorption bands listed in Table 3 (below) suggest the successful functionalization of hydrophilic magnetite nanoparticles with HSCH₂CH₂CONHNH₂ using a chemically bonded reaction via Fe—S covalent bonds and the hydrazinolysis reaction method. The characteristic absorption bands of FTIR spectra of Fe₃O₄—SCH₂CH₂CONHNH₂ in FIG. 4b are ν(N—H) (3435 cm⁻¹), ν(C—H) (2954, 2922, 2852 cm⁻¹), ν(C═O) (1626 cm⁻¹), νₐₛ(-NH⁺₃) (1401 cm⁻¹), ν(C—N) (1023 cm⁻¹) and ν(Fe—S, Fe—O) (628, 591 and 447 cm⁻¹). It may be noted from the FTIR spectra in FIG. 6b and Table 3 that most of oleylamine surfactant on the surface of the magnetite nanoparticles has been replaced by HSCH₂CH₂CONHNH₂.

TABLE 3

| Samples | IR absorption bands (cm⁻¹) | Description* |
|---|---|---|
| (a) Hydrophobic Fe₃O₄ nanoparticles | 3435 | ν(N—H ··· H) |
| | 3006 | ν(═C—H) |
| | 2954, 2922, 2852 | νₐₛ(C—H) and νₛ(C—H) of —CH₂ |
| | 1626 | ν(C═O) |
| | 1526 | Absorption band of Fe—N bond |
| | 1458, 1428 | δₐₛ(CH₃) and δₛ(CH₃) |
| | 1074 | ν(C—N) |
| | 628, 591 | Absorption bands of the Fe—O bond |
| | 447 | Absorption bands of the Fe—O bond |
| (b) Hydrophilic Fe₃O₄—SCH₂CH₂CONHNH₂ | 3435 | ν(N—H ··· H) |
| | 2954, 2922, 2852 | νₐₛ(C—H) and νₛ(C—H) of —CH₂ |
| | 1626 | ν(C═O) |
| | 1401 | ν(—NH₃⁺) |
| | 1023 | ν(C—N) |
| | 628, 591 | Absorption bands of the Fe—O and Fe—S bonds |
| | 447 | Absorption bands of the Fe—O bond |
| (c) Fe₂O₃—SCH₂CH₂CONHN═C-DOX | 3435 | ν(N—H ··· H) and ν(O—H ··· H) |
| | 2962, 2925, 2854 | νₐₛ(C—H) and νₛ(C—H) of —CH₃ and —CH₂ |
| | 1630 | ν(C═O) |
| | 1410 | ν(—NH₃⁺) |
| | 1260 | ν(═C—O—CH₃) of doxorubicin |
| | 1094, 1025 | δ(O—H) |
| | 804 | Absorption band from doxorubicin |
| | 635, 594 | Absorption bands of the Fe—O and Fe—S bonds |
| | 441 | Absorption band of the Fe—O bond |
| (d) Fe₂O₃—SCH₂CH₂CONHN═C-DOX + | 3435 | ν(N—H ··· H) and ν(O—H ··· H) |
| smart polymer | 2960, 2921, 2854 | νₐₛ(C—H) and νₛ(C—H) |
| | 1628 | ν(C═O) |
| | 1546, 1388 | ν(—CH(CH₃)₂) of the smart polymer |
| | 1404 | ν(—NH₃⁺) |
| | 1263 | ν(═C—O—CH₂) of doxorubicin |
| | 1096, 1025 | δ(O—H) |
| | 804 | Absorption band from doxorubicin |
| | 635, 588 | Absorption bands of the Fe—O and Fe—S bonds |
| | 441 | Absorption band of the Fe—O bond |

*νₛ = symmetric stretching vibration; νₐₛ = asymmetric stretching vibration; δ = bending vibration.

The Fe₃O₄—SCH₂CH₂CONHN═C-DOX (doxorubicin) conjugate obtained via reaction of hydrazide groups of the Fe₃O₄—SCH₂CH₂CONHNH₂ with carbonyl groups of the doxorubicin was confirmed by FTIR as shown in FIG. 6c. The characteristic absorption bands of 804 and 1260 cm⁻¹ (ν(=C—O—CH₃ bonds)) correspond to doxorubincin, while the other characteristic absorption bands outlined above continue to be present. The above observations suggest that doxorubicin is chemically conjugated on the surface of the functionalized magnetite nanoparticles.

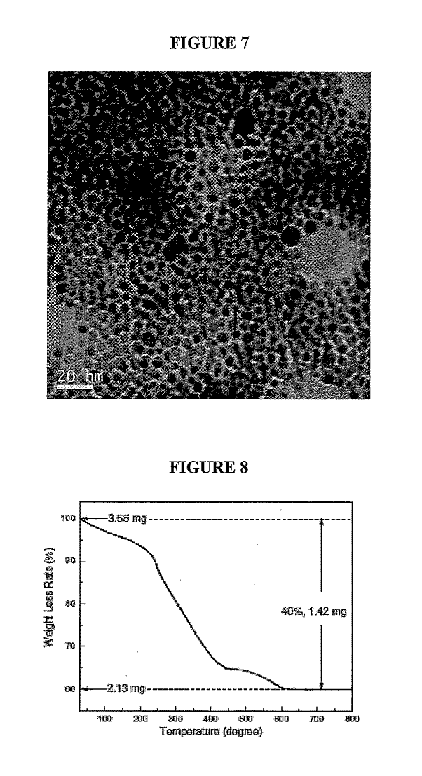

FIG. 7 is a high-magnification transmission electron micrograph of the Fe₃O₄ nanoparticles encapsulated with dextran-g-poly(NIPAAm-co-DMAAm) smart polymer. The superparamagnetic Fe₃O₄ nanoparticles encapsulated with smart polymer are nanoparticles of spherical shape with an average diameter of approximately 8 nanometers (nm). On encapsulation with polymer, the size of the nanoparticles was increased from approximately 5 nm to approximately 8 nm. The encapsulation is supported by the TGA plot presented in FIG. 8. TGA analysis was performed using a TA Instruments module, SDT 2960, with 3-5 mg of powder sample at a rate of 5° C. min⁻¹ from room temperature to 800° C. in an argon atmosphere. The mass loss of 40 wt. % on heating from room temperature to 600° C. implies that the drug carrier consisted of 60 wt. % of Fe₃O₄ nanoparticles and 40 wt. % of the organic substance, including the drug and the smart polymer.

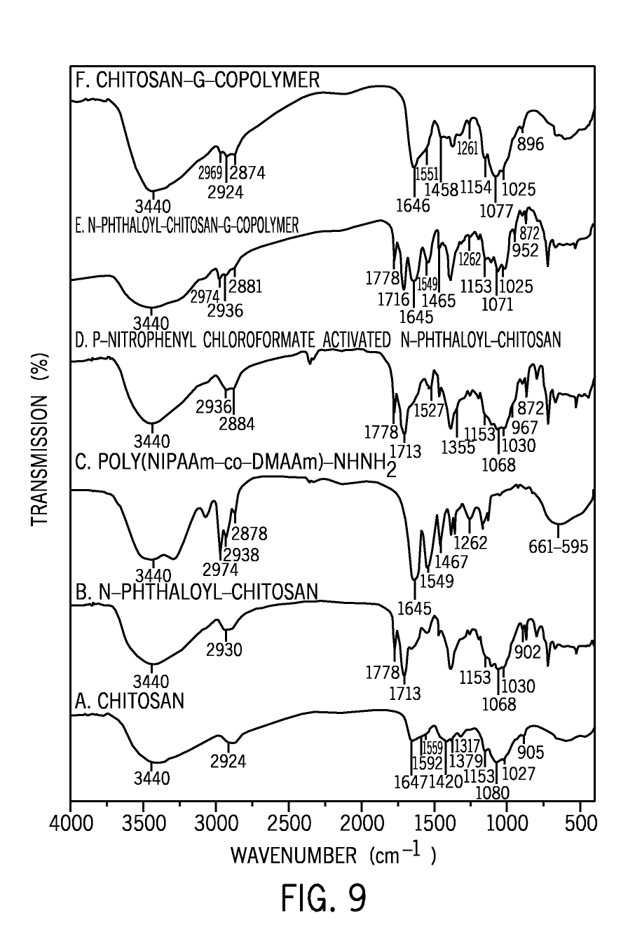

A similar FTIR study and analysis (FIG. 9 and supporting information in Table 4) of spectra confirmed the chemical synthesis of chitosan-grafted copolymer, which was subsequently used to encapsulate the doxorubicin-loaded magnetite nanoparticles. The FTIR spectrum of N-phthaloyl-chitosan (FIG. 9b), compared with that of the initial chitosan, exhibits two new absorption bands at 1778 and 1713 cm⁻¹, which are attributed to the phthalimido groups. This suggests the successful phthaloylation of chitosan. These absorption bands at 1778 and 1713 cm⁻¹ are absent in the FTIR spectrum of chitosan-g-poly(NIPAAmco-DMAAm) (FIG. 9f). This confirms the removal of the N-phthaloyl group to obtain chitosan-g-poly(NIPAAmco-DMAAm) (FIG. 9f). Characteristic absorption bands corresponding to chitosan at 3700-3200 cm⁻¹ (ν(HO . . . H)), 1096 and 1025 cm⁻¹ (δ(O—H)), and from the poly(NIPAAm-co-DMAAm) at 2960, 2921 and 2854 cm⁻¹ (νₐₛ(C—H) and νₛ(C—H) of —CH₃ and —CH₂) and 1546 and 1388 cm⁻¹ (ν(-CH(CH₃)₂)) are observed in FIG. 9d. This suggests that the functionalized magnetite nanoparticles conjugated with doxorubicin were successfully encapsulated by chitosan-g-poly(NIPAAM-co-DMAAm) biodegradable, stimuli-responsive polymer to form doxorubicin-loaded magnetite nanoparticles. The superparamagnetic nanoparticle carrier is characterized by functionalized magnetite loaded with doxorubicin and encapsulated by chitosan-gpoly(NIPAAm-co-DMAAm) biodegradable, stimuli-responsive polymer.

TABLE 4
Assignment of FT-IR spectra of dextran, 4-nitrophenyl chloroformate-activated N-phthaloylated
chitosan and poly(NIPAAm-co-DMAAm) with an amino end group presented in FIG. 5.

| Sample | IR absorption bands (cm⁻¹) | Description* |
|---|---|---|
| Chitosan | 3440 | νₛ(N—H) |
| | 2924 | ν(C—H) |
| | 1647 | ν(—C═O—) amide I |
| | 1592 | Amine |
| | 1559 | ν(N—H) amide II |
| | 1420, 1383 | δ(CH₃) |
| | 1317 | ν(—CH₃) amide III |
| | 1153, 1088, 1020 | νₐₛ(C—O—C) and νₐₛ(C—O—C) |
| | 905 | ω(CH₃) |
| N-phthaloyl-chitosan | 3440 | νₛ(N—H) |
| | 2930 | ν(C—H) |
| | 1778 | ν(C═O) imide |
| | 1713 | ν(C—N) |
| | 1153, 1068, 1030 | νₐₛ(C—O—C) and νₛ(C—O—C) |
| | 902 | ω(CH₃) |
| Poly(NIPAAm-co-DMAAm) with an amino end group | 3444, 3311 | ν(N—H ··· H) |
| | 2974, 2938, 2878 | νₐₛ(C—H) and νₛ(C—H) of —CH₃ and —CH₂ |
| | 1645 | ν(C═O) |
| | 1549 | δ(N—H) |
| | 1467 | δ(H—C—OH) |
| | 1262 | ν(C—O—CH₂—) |
| | 661-595 | ν(C—S) |
| 4-Nitrophenyl chloroformate-activated N-phthaloyl-chitosan | 3440 | ν(H—O ··· H) |
| | 2936, 2884 | ν(C—H) of —CH₂ |
| | 1778 | ν(C═O) imide |
| | 1713 | ν(C—N) |
| | 1527, 1355 | νₐₛ(N═O) and νₛ(N═O) |
| | 1153, 1068, 1030, 852 | νₐₛ(C—O—C) and νₛ(C—O—C) |
| | 967 | ν(C—N) |
| N-phthaloyl-chitosan-g-poly(NIAAm-co-DMAAm) polymer | 3440 | ν(O—H) and ν(N—H) |
| | 2974, 2936, 2881 | νₐₛ(C—H) and νₛ(C—H) |
| | 1778 | ν(C═O) imide |
| | 1713 | ν(C—N) |
| | 1645 | δ(CH₃) |
| | 1549 | δ(N—H) |
| | 1465 | δ(H—C—OH) |
| | 1262 | ν(C—O—CH₂—) |
| | 1153, 1072, 872 | νₐₛ(C—O—C) and νₛ(C—O—C) |
| | 1025 | δ(O—H) |
| Chitosan-g-poly(NIAAm-co-DMAAm) polymer | 3440 | ν(O—H) and ν(N—H) |
| | 2969, 2924, 2874 | νₐₛ(C—H) and νₛ(C—H) |
| | 1646 | δ(CH₃) |
| | 1551 | δ(N—H) |
| | 1458 | δ(H—C—OH) |
| | 1261 | |
| | 1154, 1077 | ν(C—O—CH₂—) |
| | 1025 | νₐₛ(C—O—C) and νₛ(C—O—C) |
| | 896 | δ(O—H) |
| | | ω(CH₃) |

*νₛ = symmetric stretching vibration; νₐₛ = asymmetric stretching vibration; δ = bending vibration or deformation, ω = wagging.

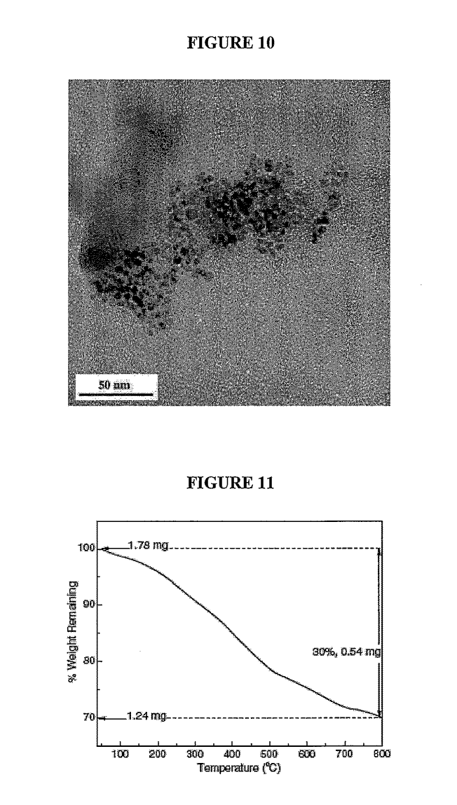

A TEM of magnetite nanoparticles encapsulated by chitosan-g-poly(NIPAAm-co-DMAAm) smart polymer is presented in FIG. 10. The magnetite nanoparticles encapsulated by the chitosan-based biodegradable, stimuli-responsive polymer have an average diameter of approximately 8 nm. Comparing the TEM micrograph in FIG. 4a with that in FIG. 10, it can be seen that, on encapsulation with the chitosan-based polymer, the size of the nanoparticles increased from approximately 5 to approximately 8 nm. This was further supported by the TGA data presented in FIG. 11. TGA analysis was performed using a TA Instruments module, SDT 2960, with 3-5 mg of powder sample at a rate of 5° C. min⁻¹ from room temperature to 800° C. in an argon atmosphere. The mass loss of approximately 30 wt. % on heating from room temperature to 800° C. implies that the drug carrier consisted of 70 wt. % magnetite and 30 wt. % organic substance, including drug and smart polymer.

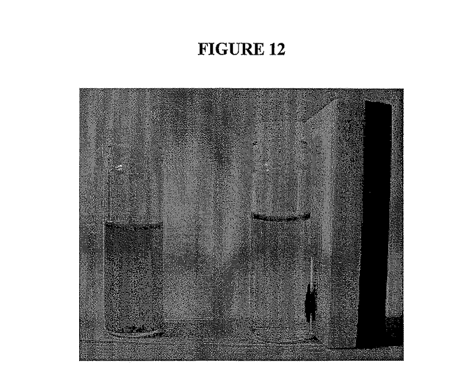

Superparamagnetism. A simple experiment was performed to illustrate that superparamagnetism was retained in the nanoparticles encapsulated with dextran-based polymer. In FIG. 12, a picture of the superparamagnetic nanoparticles attracted by an external magnet is presented. On application of an external magnetic field to a container holding the doxorubicin-loaded nanoparticles encapsulated with dextran-based smart polymer, the nanocapsules were attracted towards the magnet and became attached to the side of the container that was in close proximity to the magnet, and the dispersion became clear (right side of FIG. 12). Removal of the external magnetic field and sonication led to the recovery of the dispersion (left side of FIG. 12). This simple experiment confirmed that the superparamagnetic nanoparticles retained magnetism on encapsulation with polymer. This is an unexpected result because superparamagnetic nanoparticles encapsulated with polymers generally exhibit a lower level of magnetization in relation to bare superparamagnetic nanoparticles. When superparamagnetic nanoparticles are encapsulated with polymers, there is generally a marginal decrease in the value of superparamagnetization. This lower value is related to the overall reduction in the total superparamagnetic content in the composite nanoparticles, due to the encapsulation of the nanoparticle with the polymer. This occurs primarily due to the fact that the contribution of the magnetic ferrite content to the total mass of the nanoparticles is reduced in the polymer-encapsulated ferrite such that the overall magnetization (emu g⁻¹) of the sample is lower. Also, the non-magnetic polymer encapsulation coating can be visualized as a magnetic dead layer on the surface, influencing the magnitude of magnetization. Similar results were observed with the chitosan-based polymer encapsulated superparamagnetic nanoparticles.

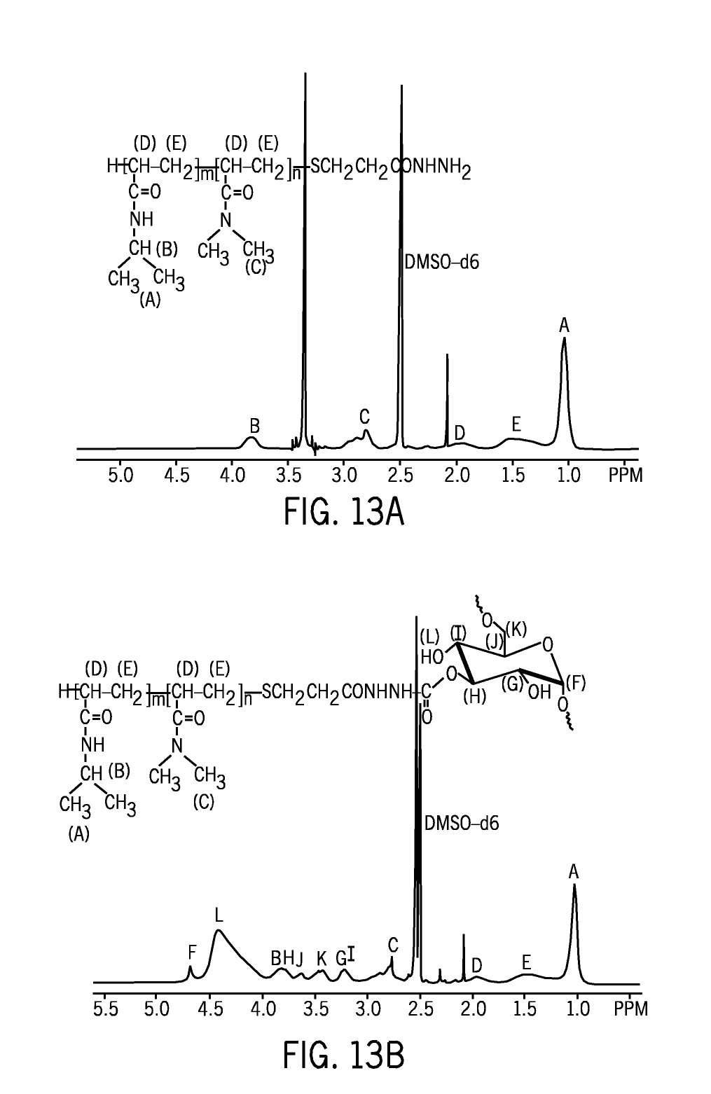

¹H NMR characterization of poly(NIPAAm-co-DMAAm) and dextran-g-poly(NIPAAm-co-DMAAm) smart polymer. Characterization of the copolymer and dextran-g-poly(NIPAAm-co-DMAAm) smart polymer. The chemical composition of the copolymer and dextran-g-poly(NIPAAm-co-DMAAm) smart polymer was examined by ¹H-nuclear magnetic resonance (NMR) using a Fourier transform-NMR spectrometer operating at 300 MHz. The ¹H NMR spectra of poly(NIPAAm-co-DMAAm) and dextran-g-poly(NIPAAm-co-DMAAm) smart polymers shown in FIG. 13 confirm the chemical composition of the smart polymers. Characteristic peaks from the different components indicating dextran (backbone) and the poly(NIPAAm-co-DMAAm) polymer (grafted chain) can be identified. Peaks a-e result from NIPAAm and DMAAm of the poly(NIPAAm-co-DMAAm) polymer, while peaks f, g, h, i, j, k and l originate from dextran. Using the standard procedure of calculation of characteristic peak integration of DMAAm and NIPAAm in ¹H NMR spectra, the molecular weight and average number of grafts in the dextran-g-poly(NIPAAm-co-DMAAm) smart polymer were estimated to be about 79,000 and 7.6, respectively.

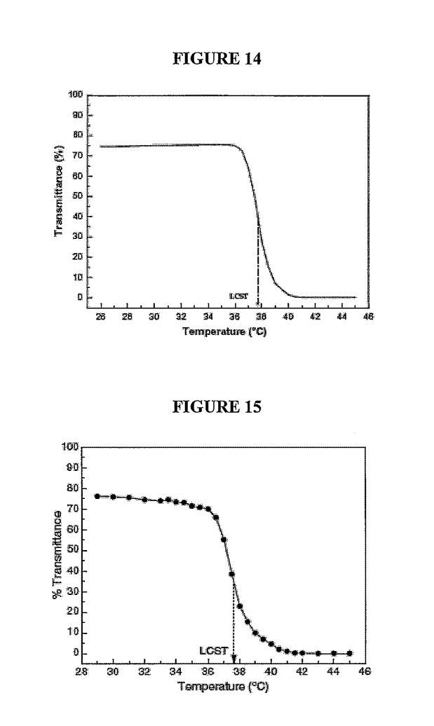

Determination of LCST of the dextran-based biodegradable, stimuli-responsive polymer. A 1.0 mg ml⁻¹ aqueous solution of the dextran-g-poly(NIPAAm-co-DMAAm) smart polymer containing 5 wt. % of PBS solution (pH 7.4) was determined using a UV-Vis spectrophotometer. The LCST measurement was performed using a UV-Vis spectrophotometer (Lauda Brinkmann, Germany) equipped with a temperature controller. The measurements were carried out by monitoring change in transmittance as a function of temperature at 500 nm wavelength. The LCST of 1.0 mg ml⁻¹ of aqueous solution of the dextran-g-poly(NIPAAm-co-DMAAm) smart polymer containing 5 wt. % of PBS solution (pH 7.4) was determined using a UV-Vis spectrophotometer (FIG. 14). The smart polymer exhibits a LCST of approximately 38° C. (ranging from 36 to 40° C.) in PBS solution. The obtained LCST of approximately 38° C. is the desired temperature for drug-controlled release because it is slightly higher than the normal physiological body temperature of 37° C. It may be noted that the LCST of the colloidal solution containing the superparamagnetic nanoparticle carrier encapsulated with the smart polymer cannot be measured using the UV-Vis spectrophotometer method because of its poor transparency. However, we believe that LCST of encapsulated magnetite nanoparticles will vary insignificantly from that of the pure smart polymer because the nanoparticles are characterized by a cross-linked smart polymer whose composition and structure are almost identical to that of the pure smart polymer. The transmittance vs. temperature behavior (FIG. 14) of functionalized magnetite nanoparticles encapsulated with a smart polymer is indicative of the nanoparticle encapsulated with stimuli-responsive polymer has a temperature-responsive triggering mechanism.

Determination of LCST of the chitosan-based biodegradable, stimuli-responsive polymer. The LCST of 1.0 mg ml⁻¹ of aqueous solution of the chitosan-g-poly(NIPAAM-co-DMAAm) biodegradable, stimuli-responsive polymer containing 5 wt. % of PBS (pH 7.4) was determined using a UV-Vis spectrophotometer (FIG. 15). The measurements were carried out by monitoring the change in transmittance as a function of temperature at 500 nm wavelength. The starting point of transmittance was low, at approximately 75%, because the solution was not very clear. The chitosan-based polymer was characterized by an LCST of approximately 38° C. in PBS and is the temperature corresponding to the mid-point of the transition regime at a transmittance value of approximately 40%. The LCST of approximately 38° C. is an appropriate temperature from the viewpoint of controlled anti-tumor agent release, because it is very close to the physiological body temperature of 37° C. The LCST of the colloidal solution containing the superparamagnetic nanoparticles encapsulated by the smart polymer could not be obtained by the UV-Vis spectrophotometer method because the solution was not very transparent. The LCST of the encapsulated magnetite nanoparticles is not expected to differ significantly from the pure chitosan-based polymer, because the nanoparticle is characterized by a crosslinked chitosan-based biodegradable, stimuli-responsive polymer whose composition and structure are similar to those of the pure chitosan-based polymer. The transmittance vs. temperature behavior (FIG. 15) of functionalized magnetite nanoparticles encapsulated by a smart polymer is indicative of a nanoparticle encapsulated with stimuli-responsive polymer having a temperature-responsive triggering mechanism.

Drug release behavior for dextran-based smart polymer encapsulated nanoparticles To examine the drug release behavior of the dextran-based polymer encapsulated carrier in PBS (pH 5.3 and 7.4), three temperatures—room temperature (20° C.) (<LCST), physiological temperature (37° C.) (approximately LCST) and low hyperthermal temperature (40° C.) (>LCST)—were selected. In each drug release experiment, 3.0 mg of the magnetite nanoparticles, conjugated with doxorubicin and encapsulated with dextran-based stimuli-responsive polymer, was sealed in a dialysis membrane tube. The dialyses tube was submerged in 10 ml of Na₂HPO₄—KH₂PO₄ buffer solution with pH of 5.3 or 7.4, which was placed in a test tube with a closer. The test tube with the closer was placed in a water bath maintained at 40° C. (>LCST), 37° C. (approximately LCST) or 20° C. (room temperature) (<LCST). The release medium (approximately 2 ml) was withdrawn at predetermined time intervals (1, 2, 3, 4, 5, 6, 7, 8, 9, 12, 24, 36 and 48 hours) and the amount of the free doxorubicin (W_{freeDOX}) in the buffer solution was quantified using Lambert-Beer law defined above. After each measurement, the withdrawn medium was returned back to the system. Since the measurement time was very short while the drug release predetermined time interval was significantly large, the influence of the returned medium on drug release during the measurement time is expected to be insignificant. To determine the effectiveness of smart polymer, the drug release of the bare nanoparticle (without the smart polymer) was carried out in an identical manner in 10 ml of Na₂HPO₄—KH₂PO₄ buffer solution with a pH of 5.3 at 40° C. (>LCST) and 20° C. (room temperature) (<LCST). All drug release experiments were repeated at least three times.

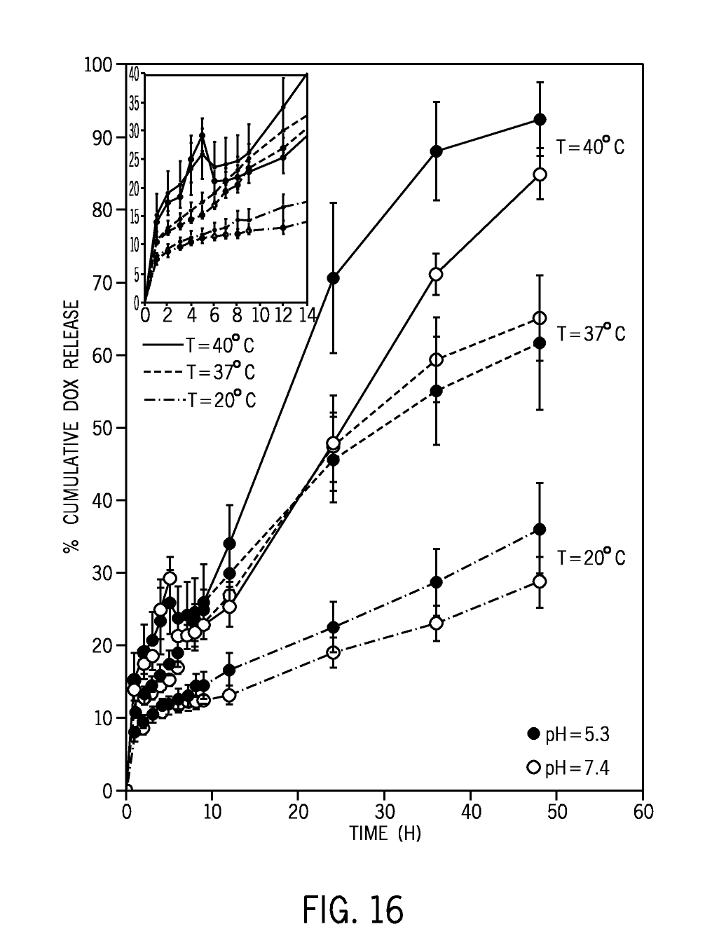

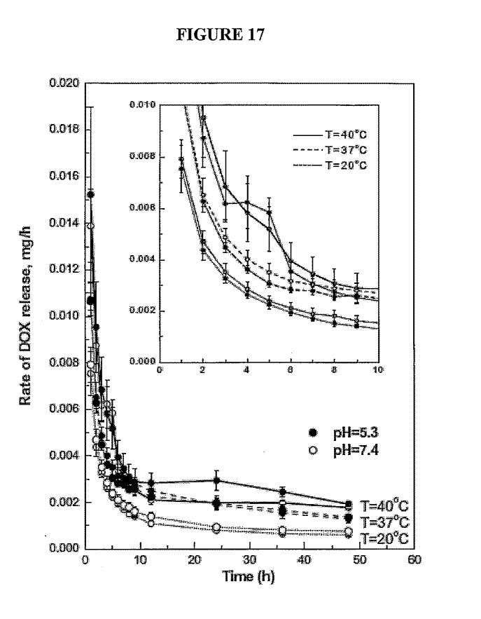

The dependence of cumulative doxorubicin release (%) and the rate of doxorubicin release (mg h⁻¹) from the drug carrier under these conditions are presented in FIGS. 16 and 17, respectively. It can be seen from FIG. 16 that at 20° C. (<LCST) the release of doxorubicin was very low (<35%) for both experimental pHs of 7.4 and 5.3. The release of doxorubicin at longer durations at pH 5.3 was greater by only 5-10% in comparison to pH 7.4. This is most likely a consequence of the acid-labile linker (hydrozone linkage). However, the drug release during the first 1-2 hours from the encapsulated carriers was relatively rapid (inset of FIGS. 16 and 17). This was followed by a slow rate of release (FIGS. 16 and 17).

In contrast to 20° C., at 40° C. (>LCST) the drug release from the encapsulated carriers at pH 5.3 and 7.4 was higher and relatively faster. Furthermore, a pulsatile release occurred within the initial 5 hours where rapid (burst) release of approximately 30% occurred. This may have been triggered by a change in the carrier's chemical environment. At longer durations, from 6 to 36 hours, at both pH 5.3 and 7.4, drug release continued to increase, followed by a nearly sustained release. In addition, the drug release at pH of 5.3 was greater than at pH of 7.4, a behavior attributed to the acid-labile linker (hydrozone linkage).

The drug release behavior in the pH 5.3 and 7.4 buffer solutions at 37° C. (approximately LCST or in the LCST range) was similar to that at both 20 and 40° C. The drug release at pH 5.3 was marginally greater than at pH 7.4.

The rate of doxorubicin release presented in FIG. 17 suggests a high release rate for the first 5 hours, followed by a sustained release rate. A similar behavior was observed at other temperatures and pHs.

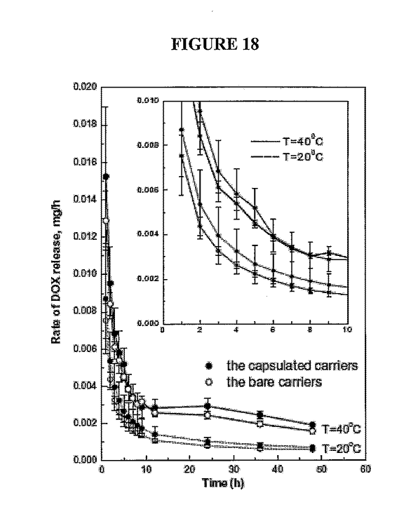

A similar conclusion can be derived from FIG. 18 as from FIG. 17, i.e. a rapid release and a rapid decrease within the initial 5 hours, followed by a sustained release. The influence of temperature and pH on rate of doxorubicin release is again similar to FIG. 17.

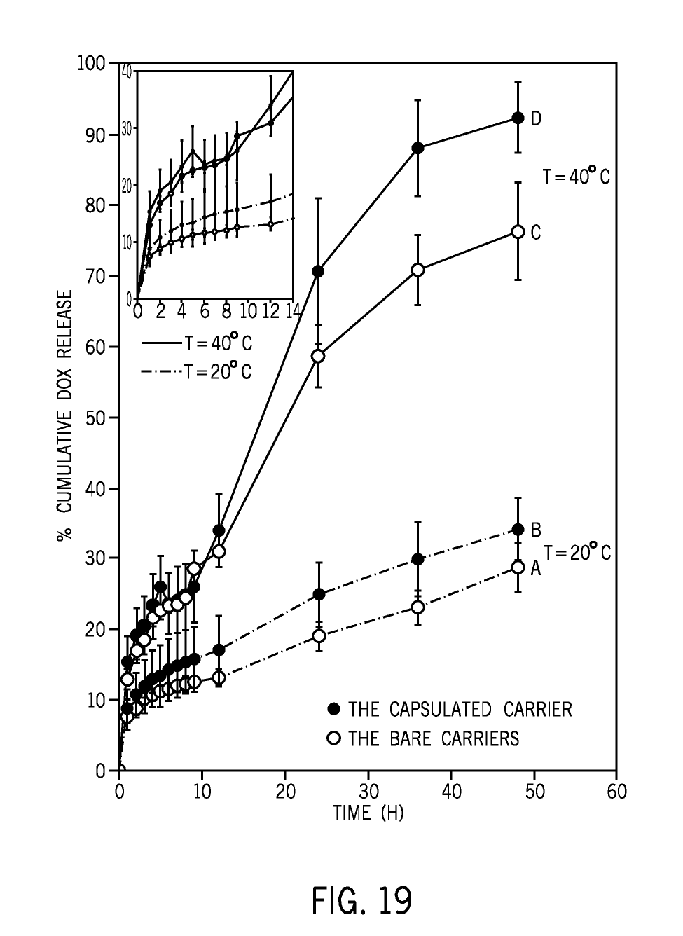

Considering that the dextran-g-poly(NIPAAm-co-DMAAm) smart polymer is thermosensitive, it can be used for regulating drug release via response to temperature change in the vicinity of LCST by swelling and deswelling. To confirm the effect of the smart polymer on drug release in response to temperature change, the cumulative doxorubicin release and the rate of doxorubicin release (mg h⁻¹) from the encapsulated superparamagnetic nanoparticles and the bare carrier without the smart polymer under identical experimental conditions (pH 5.3, 20 and 40° C.) is presented in FIGS. 19 and 18. It may be noted from FIG. 19 that the drug release is greater for the smart polymer encapsulated nanoparticle than the bare nanoparticle at both the experimental temperatures of 20 and 40° C. at longer durations (>_{—}10 hours), implying that the smart polymer is responsible for higher drug release. The doxorubicin release for durations of <10 hours corresponds to the burst release stage.

The above observations lead us to suggest that the drug release response depends on temperature and pH; temperature greater than the LCST and mild acidic medium favors drug release. Interestingly, the drug released can be controlled through small changes in temperature in the vicinity of the LCST and pH. When the temperature was below the LCST, the drug carrier is stable and drug release is slow. However, when the temperature is greater than the LCST, the smart polymer collapses such that the squeezing effect of the polymer leads to enhanced drug release. Additionally, the acid-labile linker (hydrozone linkage) promotes drug release in mildly acidic medium as compared with the neutral medium under identical experimental conditions. It should be pointed out that the above pulsatile drug release is related to intricate burst release.

Drug release behavior for chitosan-based smart polymer encapsulated nanoparticles. The drug release behavior of the chitosan-based polymer encapsulated nanoparticle in PBS (pH 5.3 and 7.4) was studied at three different temperatures (40° C. (above LCST), 37° C. (physiological temperature) and 20° C. (below LCST)) and at pHs of 5.3 and 7.4. In each experiment, 2.0 mg of the nanoparticles conjugated with doxorubicin and encapsulated by the chitosan-based biodegradable, stimuli-responsive polymer was sealed in a dialysis membrane tube. The dialysis tube was submerged in 10 ml of Na₂HPO₄—KH₂PO₄ buffer solution, pH 5.3 or 7.4, and placed in a test tube with a closer. The test tube with the closer was placed in a water bath maintained at 40° C. (>LCST) or 20° C. (room temperature) (<LCST). The release medium (approximately 2 ml) was withdrawn at predetermined time intervals (1, 2, 3, 4, 5, 6, 7, 8, 9, 12, 24, 36 and 48 hours) and the amount of the released doxorubicin (W_{free DOX}) in the buffer solution was quantified using the Lambert-Beer law defined above. After each measurement, the withdrawn medium was returned to the system. Since the measurement time was very short and the drug release predetermined time interval was significantly large, the influence of the returned medium on drug release during the measurement time is expected to be insignificant. All drug release experiments were repeated at least three times.

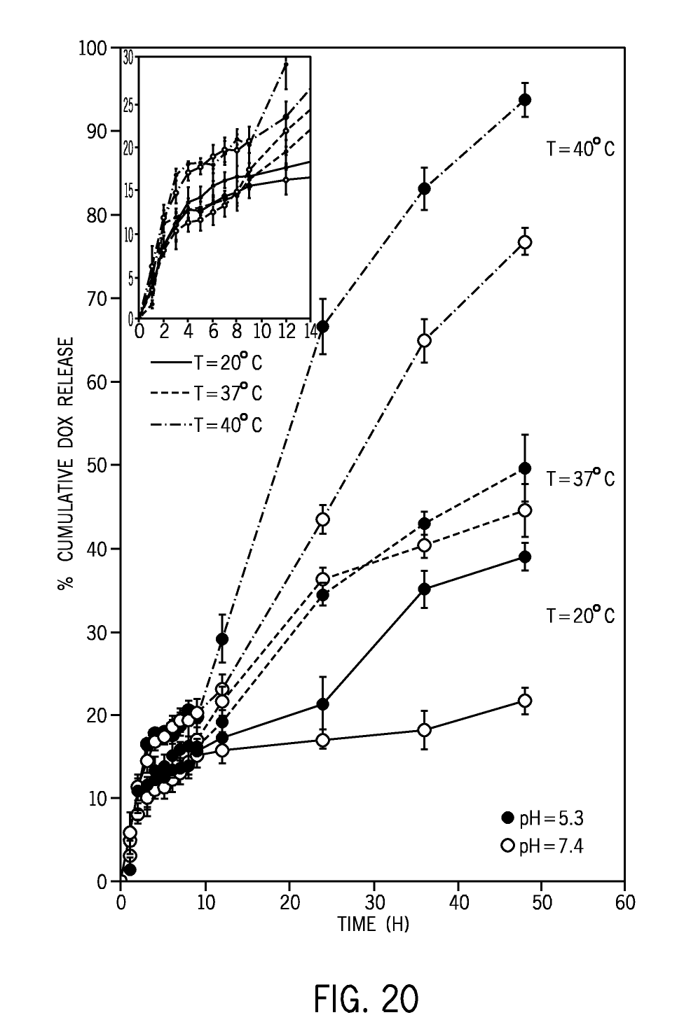

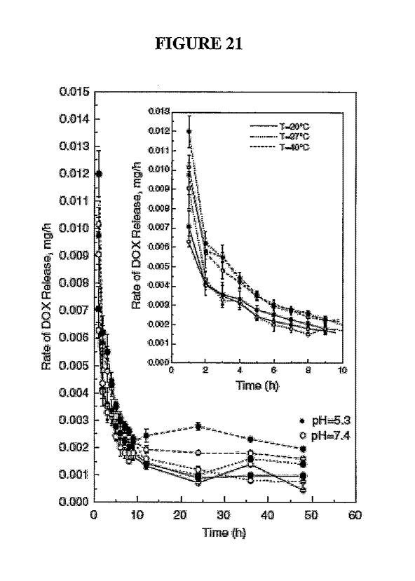

The dependence of cumulative doxorubicin release and the rate of doxorubicin release (mg h⁻¹) under these conditions are presented in FIGS. 20 and 21, respectively. At 20° C. (<LCST), the release of doxorubicin was less than approximately 40% at pH 5.3 and less than approximately 20% at pH 7.4. The release of doxorubicin at longer times at pH 5.3 was greater by 15-20% than at pH 7.4. This is most likely to be due to acid-labile linker (hydrazone linkage). However, the drug release during 1-4 hours from the nanoparticle carrier was comparatively rapid (inset of FIG. 21), followed by a slower rate of release at both the investigated pHs. The drug release behavior at 37° C. was similar to that at 40° C., but the extent of release was intermediate between 27 and 40° C.

The drug release from the nanoparticle drug carrier at 40° C. (>LCST) at both pHs was greater and faster than at the lower temperatures. Moreover, a pulsatile release occurred during the early stages (1-4 hours) such that there was a rapid release of approximately 20%. This could be because of the change in the nanoparticle's chemical environment. At longer durations, from 4 to 36 hours (at pH 5.3 and 7.4), the doxorubicin release continued to increase, before displaying a tendency to exhibit a near sustained release. Additionally, the doxorubicin release at pH 5.3 was greater than that at pH 7.4, a behavior attributed to acid-labile linker (hydrozone linkage).

From FIG. 21, the rate of doxorubicin release suggests a high release rate for the first 4 hours, followed by a sustained release rate. The behavior was similar at both the temperatures and pH.

The aforementioned observations suggests that the drug release response is dependent upon temperature and pH; a temperature greater than the LCST and a mild acidic medium are favorable for drug release. The results indicate that the release of a therapeutic agent can be controlled via small changes in temperature in the vicinity of the LCST and via pH. At temperatures less than the LCST, the nanoparticle is stable and drug release is slow. However, above the LCST, the smart polymer collapses such that the squeezing effect of the polymer encourages enhanced drug release. Additionally, the acid-labile linker (hydrozone linkage) promotes drug release in a mild acidic medium as compared with a neutral medium under identical experimental conditions. The observed initial rapid release is most likely to be related to the intricate burst release and is presently not understood.

Conjugation with folic acid. First, the carboxyl group of folic acid is activated to form NHS-folate, which further reacts with the amine tethers on the surface of the encapsulated nanoparticles. Active NHS-folate is obtained by adding NHS (0.2652 g) and dicyclohexylcarbodiimide (0.4754 g) to the solution of folic acid (1 g) in dimethylsulfoxide (50 mL). The byproduct dicyclohexylurea is removed by centrifugation. The supernatant is dialyzed against deionized water to remove DMSO. Aliquots of this solution (50 μL) are added to a 5 mL suspension of dextran-g-poly(NIPAAm-co-DMAAm) or chitosan-g-poly(NIPAAM-co-DMAAm) encapsulated drug-loaded nanoparticles. The resulting mixture is stirred at 4° C. in the dark (the reaction is light sensitive) for 16 hours during which time the NHS-folate reacts with the amine tethers on dextran-g-poly(NIPAAm-co-DMAAm) or chitosan-g-poly(NIPAAM-co-DMAAm) encapsulated drug-loaded superparamagnetic nanoparticles. After the reaction, the nanoparticles are recovered by centrifugation (15,000 rpm×20 min, 4° C.). The amount of conjugated folic acid can be determined by UV-Vis spectrophotometry by comparing the absorbance of the folic acid at 365 nm in distilled water at pH 7 with a constructed folic acid calibration curve.

There are of course other alternate embodiments which are obvious from the foregoing descriptions of the invention, which are intended to be included within the scope of the invention, as defined by the following claims.

1. A superparamagnetic nanoparticle comprising:
(a) a core having responsivity to a magnetic field, wherein said core comprises a surface;
(b) a therapeutic agent conjugated to said surface of said core; and
(c) a stimuli-responsive polymer encapsulating said core and said therapeutic agent.

2. The superparamagnetic nanoparticle in claim 1, wherein said core comprises Fe₃O₄.

3. The superparamagnetic nanoparticle in claim 2, wherein said surface of said core is functionalized with —NHNH₂.

4. The superparamagnetic nanoparticle in claim 1, wherein said stimuli-responsive polymer comprises a biodegradable polymer.

5. The superparamagnetic nanoparticle in claim 3, wherein said stimuli-responsive polymer comprises a biodegradable polymer.

6. The superparamagnetic nanoparticle in claim 1, wherein said therapeutic agent comprises doxorubicin.

7. The superparamagnetic nanoparticle in claim 5, wherein said stimuli-responsive polymer comprises dextran-g-poly(NIPAAm-co-DMAAm) conjugated with folic acid.

8. The superparamagnetic nanoparticle in claim 5, wherein the stimuli-responsive polymer comprises chitosan-g-poly(NIPAAM-co-DMAAm).

9. The superparamagnetic nanoparticle in claim 8, wherein folic acid is conjugated with said chitosan-g-poly(NIPAAM-co-DMAAm).

10. A method for making superparamagnetic nanoparticles, comprising the steps:
(a) making a nanoparticle core having responsivity to a magnetic field, said nanoparticle comprising a surface;
(b) functionalizing said surface of said nanoparticle core;
(c) conjugating a therapeutic agent to said surface of said functionalized nanoparticle core; and
(d) encapsulating said conjugated therapeutic agent and said nanoparticle core with a stimuli-responsive polymer.

11. The method for making a superparamagnetic nanoparticle in claim 10, wherein said therapeutic agent comprises doxorubicin.

12. The method for making a superparamagnetic nanoparticle in claim 10, wherein said stimuli-responsive polymer comprises a biodegradable polymer.

13. The method for making a superparamagnetic nanoparticle in claim 12, wherein said stimuli-responsive polymer comprises dextran-g-poly(NIPAAm-co-DMAAm) conjugated with folic acid.

14. The method for making a superparamagnetic nanoparticle in claim 12, wherein said stimuli-responsive polymer comprises chitosan-g-poly(NIPAAM-co-DMAAm).

15. The method for making a superparamagnetic nanoparticle in claim 14, wherein folic acid is conjugated with said chitosan-g-poly(NIPAAM-co-DMAAm).

16. A method for making a superparamagnetic nanoparticle encapsulated with a stimuli-responsive polymer, comprising the steps:
(a) making a nanoparticle core having responsivity to a magnetic field, said nanoparticle comprising a surface;
(b) functionalizing said surface of said nanoparticle core with a —NHNH₂ functional group;
(c) conjugating a therapeutic agent to said surface of said functionalized nanoparticle core; and
(d) encapsulating said conjugated therapeutic agent and said functionalized nanoparticle core with a stimuli-responsive polymer.

17. The method for making a superparamagnetic nanoparticle in claim 16, wherein said therapeutic agent comprises doxorubicin.

18. The method for making a superparamagnetic nanoparticle in claim 16, wherein said stimuli-responsive polymer comprises a biodegradable polymer.

19. The method for making a superparamagnetic nanoparticle in claim 18, wherein said stimuli-responsive polymer comprises dextran-g-poly(NIPAAm-co-DMAAm) conjugated with folic acid.

20. The method for making a superparamagnetic nanoparticle in claim 18, wherein said stimuli-responsive polymer comprises chitosan-g-poly(NIPAAM-co-DMAAm).
